(19) United States
(12) Patent Application Publication
(10) Pub. No.: US 2007/0219192 A1
(43) Pub. Date: Sep. 20, 2007

(54) Certain chemical entities, compositions, and methods

(76) Inventors: Xiangping Qian, Foster City, CA (US); Gustave Bergnes, Pacifica, CA (US); Kenneth A. Newlander, West Chester, PA (US); Duke Fitch, Spring City, PA (US); Dashyant Dhanak, West Chester, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/592,737

(22) Filed: Nov. 2, 2006

Related U.S. Application Data
(60) Provisional application No. 60/733,040, filed on Nov. 2, 2005.

(51) Int. Cl. A61K 31/5383; A61K 31/426; A61K 31/4745; A61K 31/4172; A61K 31/4184

(52) U.S. Cl. 514/230.5; 514/375; 514/394; 514/303; 514/365; 514/406; 514/396; 514/383; 544/91; 546/119; 548/307.1; 548/190; 548/335.5; 548/265.4; 548/371.4

(57) ABSTRACT

Compounds useful for treating cellular proliferative diseases and disorders by modulating the activity of one or more mitotic kinesins are disclosed.

This application claims the benefit of provisional U.S. Patent Application No. 60/733,040, filed Nov. 2, 2005, which is hereby incorporated by reference.

This invention relates to chemical entities which are inhibitors of one or more mitotic kinesins and are useful in the treatment of cellular proliferative diseases, for example cancer, hyperplasias, restenosis, cardiac hypertrophy, immune disorders, fungal disorders, and inflammation.

Among the therapeutic agents used to treat cancer are the taxanes and vinca alkaloids, which act on microtubules. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Provided is at least one chemical entity chosen from compounds of Formula I 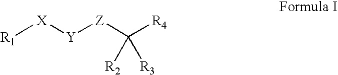
and pharmaceutically acceptable salts, solvates, chelates, non-covalent complexes, prodrugs, and mixtures thereof, wherein
R₁ is chosen from optionally substituted cycloalkyl, optionally substituted aryl, optionally substituted heterocycloalkyl, and optionally substituted heteroaryl;
X is chosen from —(CR₁₀R_{11 )}ₘ, —(CR₁₀R₁₁)ₙC(R₁₃)═C(R₁₄), —O(CR₁₀R₁₁)ₚ—, and NR₈—;
Y is chosen from a direct bond linking X and Z, —C(O)—, and —C(═N—R₉)—;
Z is chosen from —(CR₁₀R₁₁)_{q}, —(CR₁₀R₁₁)ᵣC(R₁₃)═C(R₁₄), —O(CR₁₀R₁₁)ₛ—, and NR₈—;
R₈ is chosen from —CO—R₇, hydrogen, alkoxy, optionally substituted alkyl, optionally substituted cycloalkyl, optionally substituted heterocycloalkyl, sulfonyl, optionally substituted aryl, and optionally substituted heteroaryl;
R₉ is chosen from hydrogen, alkoxy, optionally substituted alkyl, optionally substituted cycloalkyl, optionally substituted heterocycloalkyl, cyano, optionally substituted aryl, and optionally substituted heteroaryl;
R₁₀ and R₁₁ are independently chosen from hydrogen, hydroxy, optionally substituted alkyl, optionally substituted aryl, optionally substituted heterocycloalkyl, or optionally substituted cycloalkyl;
R₁₃ and R₁₄ are independently chosen from hydrogen, optionally substituted alkyl, optionally substituted aryl, optionally substituted heterocycloalkyl, or optionally substituted cycloalkyl;
m is chosen from 0, 1, and 2;
n is chosen from 0 and 1;
p is chosen from 0, 1, and 2;
q is chosen from 0, 1, and 2;
r is chosen from 0 and 1;
s is chosen from 0, 1, and 2;
R₂ is chosen from hydrogen, hydroxy, optionally substituted alkoxy, optionally substituted amino, optionally substituted heterocycloalkyl, optionally substituted cycloalkyl, and optionally substituted alkyl;
R₃ and R₄ are independently chosen from hydrogen, optionally substituted alkyl, optionally substituted alkoxycarbonyl, optionally substituted amino, aminocarbonyl, optionally substituted aryl, optionally substituted heteroaryl, optionally substituted heterocycloalkyl, and optionally substituted cycloalkyl; or
R₂ and R₄, taken together with the carbon to which they are bound, form an optionally substituted 3 to 7-membered ring which optionally includes one, two, or three heteroatoms chosen from O, N, and S; or
R₂ and R₃, taken together with the carbon to which they are bound, form an optionally substituted 3 to 7-membered ring which optionally includes one, two, or three heteroatoms chosen from O, N, and S; and
R₇ is chosen from optionally substituted lower alkyl, optionally substituted aryl, hydroxy, optionally substituted amino, optionally substituted aralkoxy, or optionally substituted alkoxy,
provided that when Y is —C(O)—, and Z is NR₈, then X is not —(CR₁₀R₁₁)ₘ wherein m is 0; and provided that if Y is a direct bond linking X and Z and X is —(CR₁₀R₁₁)ₘ wherein m is 0, then Z is not —(CR₁₀R₁₁)_{q} wherein q is 0.

Also provided is a composition comprising a pharmaceutical excipient and at least one chemical entity described herein.

Also provided is a method of modulating CENP-E kinesin activity which comprises contacting said kinesin with an effective amount of at least one chemical entity described herein.

Also provided is a method of inhibiting CENP-E which comprises contacting said kinesin with an effective amount of at least one chemical entity described herein.

Also provided is a method for the treatment of a cellular proliferative disease comprising administering to a subject in need thereof at least one chemical entity described herein.

Also provided is a method for the treatment of a cellular proliferative disease comprising administering to a subject in need thereof a composition comprising a pharmaceutical excipient and at least one chemical entity described herein.

Also provided is the use, in the manufacture of a medicament for treating cellular proliferative disease, of at least one chemical entity described herein.

Also provided is the use of at least one chemical entity described herein for the manufacture of a medicament for treating a disorder associated with CENP-E kinesin activity.

As used in the present specification, the following words and phrases are generally intended to have the meanings as set forth below, except to the extent that the context in which they are used indicates otherwise.

As used herein, when any variable occurs more than one time in a chemical formula, its definition on each occurrence is independent of its definition at every other occurrence. In accordance with the usual meaning of “a” and “the” in patents, reference, for example, to “a” kinase or “the” kinase is inclusive of one or more kinases.

Formula I includes all subformulae thereof. For example Formula I includes compounds of Formula II.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

“Alkyl” encompasses straight chain and branched chain having the indicated number of carbon atoms, usually from 1 to 20 carbon atoms, for example 1 to 8 carbon atoms, such as 1 to 6 carbon atoms. For example C₁-C₆ alkyl encompasses both straight and branched chain alkyl of from 1 to 6 carbon atoms. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is another subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀

alkylene indicates a covalent bond and C₁ alkylene is a methylene group. When an alkyl residue having a specific number of carbons is named, all geometric combinations having that number of carbons are intended to be encompassed; thus, for example, “butyl” is meant to include n-butyl, sec-butyl, isobutyl and t-butyl; “propyl” includes n-propyl and isopropyl. “Lower alkyl” refers to alkyl groups having one to four carbons.

“Alkenyl” refers to an unsaturated branched or straight-chain alkyl group having at least one carbon-carbon double bond derived by the removal of one hydrogen atom from a single carbon atom of a parent alkene. The group may be in either the cis or trans configuration about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl buta-1,3-dien-1-yl, buta-1,3-dien-2-yl, cyclobut-1-en-1-yl, cyclobut-1-en-3-yl, cyclobuta-1,3-dien-1-yl; and the like. In certain embodiments, an alkenyl group has from 2 to 20 carbon atoms and in other embodiments, from 2 to 6 carbon atoms.

“Alkynyl” refers to an unsaturated branched or straight-chain alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 3 to 6 carbon atoms.

“Cycloalkyl” indicates a non-aromatic carbocyclic ring, usually having from 3 to 7 ring carbon atoms. The ring may be saturated or have one or more carbon-carbon double bonds. Examples of cycloalkyl groups include cyclopropyl, cyclobutyl, cyclopentyl, cyclopentenyl, cyclohexyl, and cyclohexenyl, as well as bridged and caged saturated ring groups such as norbornane.

By “alkoxy” is meant an alkyl group of the indicated number of carbon atoms attached through an oxygen bridge such as, for example, methoxy, ethoxy, propoxy, isopropoxy, n-butoxy, sec-butoxy, tert-butoxy, pentyloxy, 2-pentyloxy, isopentyloxy, neopentyloxy, hexyloxy, 2-hexyloxy, 3-hexyloxy, 3-methylpentyloxy, and the like. Alkoxy groups will usually have from 1 to 7 carbon atoms attached through the oxygen bridge. “Lower alkoxy” refers to alkoxy groups having one to four carbons.

“Mono- and di-alkylcarboxamide” encompasses a group of the formula—(C═O)NRₐR_{b} where Rₐ and R_{b} are independently chosen from hydrogen and alkyl groups of the indicated number of carbon atoms, provided that Rₐ and R_{b} are not both hydrogen.

“Acyl” refers to the groups (alkyl)-C(O)—; (cycloalkyl)-C(O)—; (aryl)-C(O)—; (heteroaryl)-C(O)—; and (heterocycloalkyl)-C(O)—, wherein the group is attached to the parent structure through the carbonyl functionality and wherein alkyl, cycloalkyl, aryl, heteroaryl, and heterocycloalkyl are as described herein. Acyl groups have the indicated number of carbon atoms, with the carbon of the keto group being included in the numbered carbon atoms. For example a C₂ acyl group is an acetyl group having the formula CH₃(C═O)—.

By “alkoxycarbonyl” is meant a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker.

By “amino” is meant the group —NH₂.

“Mono- and di-(alkyl)amino” encompasses secondary and tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino.

The term “aminocarbonyl” refers to the group —CONR^{b}R^{c}, where
R^{b} is chosen from H, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c} taken together with the nitrogen to which they are bound, form an optionally substituted 5- to 7-membered nitrogen-containing heterocycloalkyl which optionally includes 1 or 2 additional heteroatoms selected from O, N, and S in the heterocycloalkyl ring;
where each substituted group is independently substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

“Aryl” encompasses:
6-membered carbocyclic aromatic rings, for example, benzene;
bicyclic ring systems wherein at least one ring is carbocyclic and aromatic, for example, naphthalene, indane, and tetralin; and
tricyclic ring systems wherein at least one ring is carbocyclic and aromatic, for example, fluorene.
   For example, aryl includes 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

The term “aryloxy” refers to the group —O-aryl.

“Carbamimidoyl” refers to the group —C(═NH)—NH₂.

“Substituted carbamimidoyl” refers to the group —C(═NR^{e})—NR^{f}R^{g} where R^{e}, R^{f}, and R^{g} is independently chosen from: hydrogen optionally substituted alkyl, optionally substituted cycloalkyl, optionally substituted aryl, optionally substituted heteroaryl, and optionally substituted heterocycloalkyl, provided that at least one of R^{e}, R^{f}, and R^{g} is not hydrogen and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c},—NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where R^{a} is chosen from optionally substituted C₁-C₆ alkyl, optionally substituted aryl, and optionally substituted heteroaryl;
R^{b} is chosen from H, optionally substituted C₁-C₆ alkyl, optionally substituted aryl, and optionally substituted heteroaryl; and
R^{c} is independently chosen from hydrogen and optionally substituted C₁-C₄ alkyl; or R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂ NH(phenyl), —NHSO₂(C₁-C₄ alkyl), —NHSO₂(phenyl), and —NHSO₂(C₁-C₄ haloalkyl).

The term “halo” includes fluoro, chloro, bromo, and iodo, and the term “halogen” includes fluorine, chlorine, bromine, and iodine.

“Haloalkyl” indicates alkyl as defined above having the specified number of carbon atoms, substituted with I or more halogen atoms, up to the maximum allowable number of halogen atoms. Examples of haloalkyl include, but are not limited to, trifluoromethyl, difluoromethyl, 2-fluoroethyl, and penta-fluoroethyl.

“Heteroaryl” encompasses:
5- to 7-membered aromatic, monocyclic rings containing one or more, for example, from 1 to 4, or in certain embodiments, from 1 to 3, heteroatoms chosen from N, O, and S, with the remaining ring atoms being carbon;
bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring; and
tricyclic heterocycloalkyl rings containing one or more, for example, from 1 to 5, or in certain embodiments, from 1 to 4, heteroatoms chosen from N, O, and S, with the remaining ring atoms being carbon and wherein at least one heteroatom is present in an aromatic ring.
   For example, heteroaryl includes a 5- to 7-membered heterocycloalkyl, aromatic ring fused to a 5- to 7-membered cycloalkyl or heterocycloalkyl ring. For such fused, bicyclic heteroaryl ring systems wherein only one of the rings contains one or more heteroatoms, the point of attachment may be at either ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridazinyl, triazolyl, quinolinyl, pyrazolyl, and 5,6,7,8-tetrahydroisoquinolinyl. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl, cycloalkyl, or heterocycloalkyl, as defined herein

Substituted heteroaryl also includes ring systems substituted with one or more oxide (—O⁻) substituents, such as pyridinyl N-oxides.

By “heterocycloalkyl” is meant a single, non-aromatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. The ring may be saturated or have one or more carbon-carbon double bonds. Suitable heterocycloalkyl groups include, for example (as numbered from the linkage position assigned priority 1), 2-pyrrolidinyl, 2,4-imidazolidinyl, 2,3-pyrazolidinyl, 2-piperidyl, 3-piperidyl, 4-piperidyl, and 2,5-piperizinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo (=0) or oxide (—O⁻) substituents, such as piperidinyl N-oxide, morpholinyl-N-oxide, 1-oxo-1-thiomorpholinyl and 1,1-dioxo-1-thiomorpholinyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteratoms independently selected from oxygen, sulfur, and nitrogen and is not aromatic.

As used herein, “modulation” refers to a change in activity as a direct or indirect response to the presence of compounds of Formula I, relative to the activity of in the absence of the compound. The change may be an increase in activity or a decrease in activity, and may be due to the direct interaction of the compound with the kinesin, or due to the interaction of the compound with one or more other factors that in turn affect kinesin activity. For example, the presence of the compound may, for example, increase or decrease kinesin activity by directly binding to the kinesin, by causing (directly or indirectly) another factor to increase or decrease the kinesin activity, or by (directly or indirectly) increasing or decreasing the amount of kinesin present in the cell or organism.

The term “sulfanyl” includes the groups: —S-(optionally substituted (C₁-C₆)alkyl), —S-(optionally substituted aryl), —S-(optionally substituted heteroaryl), and —S-(optionally substituted heterocycloalkyl). Hence, sulfanyl includes the group C₁-C₆ alkylsulfanyl.

The term “sulfinyl” includes the groups: —S(O)-(optionally substituted (C₁-C₆)alkyl), —S(O)-optionally substituted aryl), —S(O)-optionally substituted heteroaryl), —S(O)-(optionally substituted heterocycloalkyl); and —S(O)-(optionally substituted amino).

The term “sulfonyl” includes the groups: —S(O₂)-(optionally substituted (C₁-C₆)alkyl), —S(O₂)-(optionally substituted aryl), —S(O₂)-(optionally substituted heteroaryl), —S(O₂)-(optionally substituted heterocycloalkyl), and —S(O₂)-(optionally substituted amino).

The term “substituted”, as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

The terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where R^{a} is chosen from optionally substituted C₁-C₆ alkyl, optionally substituted cycloalkyl, optionally substituted heterocycloalkyl, optionally substituted alkenyl, optionally substituted alkynyl, optionally substituted aryl, and optionally substituted heteroaryl;
R^{b} is chosen from hydrogen, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted acyl” refers to the groups (substituted alkyl)-C(O)—; (substituted cycloalkyl)-C(O)—; (substituted aryl)-C(O)—; (substituted heteroaryl)-C(O)—; and (substituted heterocycloalkyl)-C(O)—, wherein the group is attached to the parent structure through the carbonyl functionality and wherein substituted alkyl, cycloalkyl, aryl, heteroaryl, and heterocycloalkyl, refer respectively to alkyl, cycloalkyl, aryl, heteroaryl, and heterocycloalkyl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b} R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where R^{a} is chosen from optionally substituted C₁-C₆ alkyl, optionally substituted alkenyl, optionally substituted alkynyl, optionally substituted aryl, and optionally substituted heteroaryl;
R^{b} is chosen from H, optionally substituted C₁-C₆ alkyl, optionally substituted cycloalkyl, optionally substituted heterocycloalkyl, optionally substituted aryl, and optionally substituted heteroaryl; and
R_{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ —SO₂NH(phenyl), —NHSO₂(C₁-C₄ alkyl), —NHSO₂(phenyl), and —NHSO₂(C₁-C₄ haloalkyl).

The term “substituted alkoxy” refers to alkoxy wherein the alkyl constituent is substituted (i.e., —O-(substituted alkyl)) wherein “substituted alkyl” refers to alkyl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where R^{a} is chosen from optionally substituted C₁-C₆ alkyl, optionally substituted alkenyl, optionally substituted alkynyl, optionally substituted aryl, and optionally substituted heteroaryl;
R^{b} is chosen from H, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl). In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of 2-20, such as 2-10, and for example, 2-5. Another substituted alkoxy group is hydroxyalkoxy or —OCH₂(CH₂)_{y}OH, where y is an integer of 1-10, such as 1-4.

The term “substituted alkoxycarbonyl” refers to the group (substituted alkyl)-O—C(O)— wherein the group is attached to the parent structure through the carbonyl functionality and wherein substituted refers to alkyl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where R^{a} is chosen from optionally substituted C₁-C₆ alkyl, optionally substituted alkenyl, optionally substituted alkynyl, optionally substituted aryl, and optionally substituted heteroaryl;
R^{b} is chosen from H, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted amino” refers to the group —NHR^{d} or —NR^{d}R^{e} wherein R^{d} is chosen from: hydroxy, optionally substitued alkoxy, optionally substituted alkyl, optionally substituted cycloalkyl, optionally substituted acyl, optionally substituted carbamimidoyl, optionally substituted aminocarbonyl, optionally substituted aryl, optionally substituted heteroaryl, optionally substituted heterocycloalkyl, optionally substituted alkoxycarbonyl, sulfinyl and sulfonyl, and wherein R^{e} is chosen from: optionally substituted alkyl, optionally substituted cycloalkyl, optionally substituted aryl, optionally substituted heteroaryl, and optionally substituted heterocycloalkyl, and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where Rₐ is chosen from optionally substituted C₁-C₆ alkyl, optionally substituted alkenyl, optionally substituted alkynyl, optionally substituted aryl, and optionally substituted heteroaryl;
R^{b} is chosen from H, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl); and
wherein optionally substituted acyl, optionally substituted alkoxycarbonyl, sulfinyl and sulfonyl are as defined herein.

The term “substituted amino” also refers to N-oxides of the groups —NHR^{d}and NR^{d}R^{d} each as described above. N-oxides can be prepared by treatment of the corresponding amino group with, for example, hydrogen peroxide or m-chloroperoxybenzoic acid. The person skilled in the art is familiar with reaction conditions for carrying out the N-oxidation.

Compounds of Formula I include, but are not limited to, optical isomers of compounds of Formula I, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formula I include Z- and E-forms (or cis- and trans- forms) of compounds with carbon-carbon double bonds. Where compounds of Formula I exists in various tautomeric forms, chemical entities of the present invention include all tautomeric forms of the compound.

Chemical entities of the present invention include, but are not limited to compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, solvates, chelates, non-covalent complexes, prodrugs, and mixtures.

“Pharmaceutically acceptable salts” include, but are not limited to salts with inorganic acids, such as hydrochloride, phosphate, diphosphate, hydrobromide, sulfate, sulfinate, nitrate, and like salts; as well as salts with an organic acid, such as malate, maleate, fumarate, tartrate, succinate, citrate, lactate, methanesulfonate, p-toluenesulfonate, 2-hydroxyethylsulfonate, benzoate, salicylate, stearate, and alkanoate such as acetate, HOOC-(CH₂)ₙ—COOH where n is 0-4, and like salts. Similarly, pharmaceutically acceptable cations include, but are not limited to sodium, potassium, calcium, aluminum, lithium, and ammonium.

In addition, if the compound of Formula I is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As noted above, prodrugs also fall within the scope of chemical entities, for example ester or amide derivatives of the compounds of Formula I. The term “prodrugs” includes any compounds that become compounds of Formula I when administered to a patient, e.g., upon metabolic processing of the prodrug. Examples of prodrugs include, but are not limited to, acetate, formate, phosphate, and benzoate and like derivatives of functional groups (such as alcohol or amine groups) in the compounds of Formula I.

The term “solvate” refers to the chemical entity formed by the interaction of a solvent and a compound. Suitable solvates are pharmaceutically acceptable solvates, such as hydrates, including monohydrates and hemi-hydrates.

The term “chelate” refers to the chemical entity formed by the coordination of a compound to a metal ion at two (or more) points.

The term “non-covalent complex” refers to the chemical entity formed by the interaction of a compound and another molecule wherein a covalent bond is not formed between the compound and the molecule. For example, complexation can occur through van der Waals interactions, hydrogen bonding, and electrostatic interactions (also called ionic bonding).

The term “active agent” is used to indicate a chemical entity which has biological activity. In certain embodiments, an “active agent” is a compound having pharmaceutical utility. For example an active agent may be an anti-cancer therapeutic.

By “significant” is meant any detectable change that is statistically significant in a standard parametric test of statistical significance such as Student's T-test, where p<0.05.

The term “antimitotic” refers to a drug for inhibiting or preventing mitosis, for example, by causing metaphase arrest. Some antitumour drugs block proliferation and are considered antimitotics.

The term “therapeutically effective amount” of a chemical entity of this invention means an amount effective, when administered to a human or non-human patient, to provide a therapeutic benefit such as amelioration of symptoms, slowing of disease progression, or prevention of disease e.g., a therapeutically effective amount may be an amount sufficient to decrease the symptoms of a disease responsive to CENP-E inhibition. In some embodiments, a therapeutically effective amount is an amount sufficient to reduce cancer symptoms. In some embodiments a therapeutically effective amount is an amount sufficient to decrease the number of detectable cancerous cells in an organism, detectably slow, or stop the growth of a cancerous tumor. In some embodiments, a therapeutically effective amount is an amount sufficient to shrink a cancerous tumor.

The term “inhibition” indicates a significant decrease in the baseline activity of a biological activity or process. “Inhibition of CENP-E activity” refers to a decrease in CENP-E activity as a direct or indirect response to the presence of at least one chemical entity described herein, relative to the activity of CENP-E in the absence of the at least one chemical entity. The decrease in activity may be due to the direct interaction of the chemical entity with CENP-E, or due to the interaction of the chemical entity(ies) described herein with one or more other factors that in turn affect CENP-E activity. For example, the presence of the chemical entity(ies) may decrease CENP-E activity by directly binding to CENP-E, by causing (directly or indirectly) another factor to decrease CENP-E activity, or by (directly or indirectly) decreasing the amount of CENP-E present in the cell or organism.

A “disease responsive to CENP-E inhibition” is a disease in which inhibiting CENP-E provides a therapeutic benefit such as an amelioration of symptoms, decrease in disease progression, prevention or delay of disease onset, or inhibition of aberrant activity of certain cell-types.

“Treatment” or “treating” means any treatment of a disease in a patient, including:
a) preventing the disease, that is, causing the clinical symptoms of the disease not to develop;
b) inhibiting the disease;
c) slowing or arresting the development of clinical symptoms; and/or
d) relieving the disease, that is, causing the regression of clinical symptoms.

“Patient” refers to an animal, such as a mammal, that has been or will be the object of treatment, observation or experiment. The methods of the invention can be useful in both human therapy and veterinary applications. In some embodiments, the patient is a mammal; in some embodiments the patient is human; and in some embodiments the patient is chosen from cats and dogs.

The compounds of Formula I can be named and numbered in the manner described below. For example, using nomenclature software, such as Pipeline Pilot or Nomenclator™ available from ChemInnovation Software, Inc., the compound: 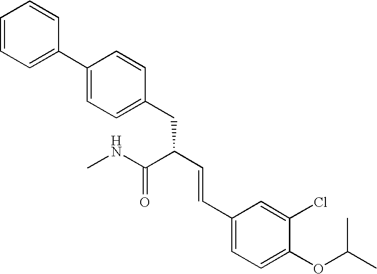
can be named (3E)(2R)-4-[3-chloro-4-(methylethoxy)phenyl]-N-methyl-2-[(4-phenylphenyl)methyl]but-3-enamide. If that same compound is named with structure=name algorithm of ChemDraw Ultra 9.0, the name is (R,E)-2-(biphenyl-4-ylmethyl)-4-(3-chloro-4-isopropoxyphenyl)-N-methylbut-3-enamide. The structures in the Example section below were named with ChemDraw. The other compounds, for example those recited in the claims below, were named with the ChemInnovation Software.

The present invention is directed to a class of novel chemical entities that are inhibitors of one or more mitotic kinesins. According to some embodiments, the chemical entities described herein inhibit the mitotic kinesin, CENP-E, particularly human CENP-E. CENP-E is a plus end-directed microtubule motor essential for achieving metaphase chromosome alignment. CENP-E accumulates during interphase and is degraded following completion of mitosis. Microinjection of antibody directed against CENP-E or overexpression of a dominant negative mutant of CENP-E causes mitotic arrest with prometaphase chromosomes scattered on a bipolar spindle. The tail domain of CENP-E mediates localization to kinetochores and also interacts with the mitotic checkpoint kinase hBubR1. CENP-E also associates with active forms of MAP kinase. Cloning of human (Yen, et al., Nature, 359(6395):536-9 (1992)) CENP-E has been reported. In Thrower, et al., EMBO J., 14:918-26 (1995) partially purified native human CENP-E was reported on. Moreover, the study reported that CENP-E was a minus end-directed microtubule motor. Wood, et al., Cell, 91:357-66 (1997)) discloses expressed Xenopus CENP-E in E. coli and that XCENP-E has motility as a plus end directed motor in vitro. CENP-E See, PCT Publication No. WO 99/13061, which is incorporated herein by reference.

In some embodiments, the chemical entities inhibit the mitotic kinesin, CENP-E, as well as modulating one or more of the human mitotic kinesins selected from HSET (see, U.S. Pat. No. 6,361,993, which is incorporated herein by reference); MCAK (see, U.S. Pat. No. 6,331,424, which is incorporated herein by reference); RabK-6 (see U.S. Pat. No. 6,544,766, which is incorporated herein by reference); Kif4 (see, U.S. Pat. No. 6,440,684, which is incorporated herein by reference); MKLP1 (see, U.S. Pat. No. 6,448,025, which is incorporated herein by reference); Kif15 (see, U.S. Pat. No. 6,355,466, which is incorporated herein by reference); Kid (see, U.S. Pat. No. 6,387,644, which is incorporated herein by reference); Mpp1, CMKrp, KinI-3 (see, U.S. Pat. No. 6,461,855, which is incorporated herein by reference); Kip3a (see, PCT Publication No. WO 01/96593, which is incorporated herein by reference); Kip3d (see, U.S. Pat. No. 6,492,151, which is incorporated herein by reference); and KSP (see, U.S. Pat. No. 6,617,115, which is incorporated herein by reference).

The methods of inhibiting a mitotic kinesin comprise contacting an inhibitor of the invention with one or more mitotic kinesin, particularly a human kinesin; or fragments and variants thereof. The inhibition can be of the ATP hydrolysis activity of the mitotic kinesin and/or the mitotic spindle formation activity, such that the mitotic spindles are disrupted.

The present invention provides inhibitors of one or more mitotic kinesins, in particular, one or more human mitotic kinesins, for the treatment of disorders associated with cell proliferation. The chemical entities compositions and methods described herein can differ in their selectivity and are used to treat diseases of cellular proliferation, including, but not limited to cancer, hyperplasias, restenosis, cardiac hypertrophy, immune disorders, fungal disorders and inflammation.

Provided is at least one chemical entity chosen from compounds of Formula I 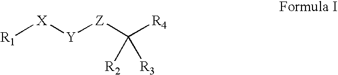
and pharmaceutically acceptable salts, solvates, chelates, non-covalent complexes, prodrugs, and mixtures thereof, wherein
R₁ is chosen from optionally substituted cycloalkyl, optionally substituted aryl, optionally substituted heterocycloalkyl, and optionally substituted heteroaryl;
X is chosen from —(CR₁₀R₁₁)ₘ, —(CR₁₀R₁₁)ₙC(R₁₃)═C(R₁₄), —O(CR₁₀R₁₁)ₚ—, and NR₈—;
Y is chosen from a direct bond linking X and Z, —C(O)—, and —C(═N—R₉)—;
Z is chosen from —(CR₁₀R₁₁)_{q}, —(CR₁₀R₁₁)ᵣC(R₁₃)═C(R₁₄), —O(CR₁₀R₁₁)ₛ—, and NR₈—;
R₈ is chosen from —CO—R₇, hydrogen, alkoxy, optionally substituted alkyl, optionally substituted cycloalkyl, optionally substituted heterocycloalkyl, sulfonyl, optionally substituted aryl, and optionally substituted heteroaryl;
R₉ is chosen from hydrogen, alkoxy, optionally substituted alkyl, optionally substituted cycloalkyl, optionally substituted heterocycloalkyl, cyano, optionally substituted aryl, and optionally substituted heteroaryl;
R₁₀ and R₁₁ are independently chosen from hydrogen, hydroxy, optionally substituted alkyl, optionally substituted aryl, optionally substituted heterocycloalkyl, or optionally substituted cycloalkyl;
R₁₃ and R₁₄ are independently chosen from hydrogen, optionally substituted alkyl, optionally substituted aryl, optionally substituted heterocycloalkyl, or optionally substituted cycloalkyl;
m is chosen from 0, 1, and 2;
n is chosen from 0 and 1;
p is chosen from 0, 1, and 2;
q is chosen from 0, 1, and 2;
r is chosen from 0 and 1;
s is chosen from 0, 1, and 2;
R₂ is chosen from hydrogen, hydroxy, optionally substituted alkoxy, optionally substituted amino, optionally substituted heterocycloalkyl, optionally substituted cycloalkyl, and optionally substituted alkyl;
R₃ and R₄ are independently chosen from hydrogen, optionally substituted alkyl, optionally substituted alkoxycarbonyl, optionally substituted amino, aminocarbonyl, optionally substituted aryl, optionally substituted heteroaryl, optionally substituted heterocycloalkyl, and optionally substituted cycloalkyl; or
R₂ and R₄, taken together with the carbon to which they are bound, form an optionally substituted 3 to 7-membered ring which optionally includes one, two, or three heteroatoms chosen from O, N, and S; or
R₂ and R₃, taken together with the carbon to which they are bound, form an optionally substituted 3 to 7-membered ring which optionally includes one, two, or three heteroatoms chosen from O, N, and S; and
R₇ is chosen from optionally substituted lower alkyl, optionally substituted aryl, hydroxy, optionally substituted amino, optionally substituted aralkoxy, or optionally substituted alkoxy,
provided that when Y is —C(O)—, and Z is NR₈, then X is not —(CR₁₀R₁₁)ₘ wherein m is 0; and provided that if Y is a direct bond linking X and Z and X is —(CR₁₀R₁₁)ₘ wherein m is 0, then Z is not —(CR₁₀R₁₁)_{q} wherein q is 0.

In some embodiments, R₁ is optionally substituted aryl. In some embodiments, R₁ is optionally substituted phenyl. In some embodiments, R₁ is phenyl substituted with one, two or three groups independently selected from optionally substituted heterocycloalkyl, optionally substituted alkyl, sulfonyl, halo, optionally substituted amino, sulfanyl, optionally substituted alkoxy, optionally substituted aryloxy, optionally substituted heteroaryloxy, acyl, hydroxy, nitro, cyano, optionally substituted aryl, and optionally substituted heteroaryl. In some embodiments, R₁ is chosen from 3-halo-4-isopropoxy-phenyl, 3-cyano-4-isopropoxy-phenyl, 3-halo-4-((R)-1,1,1-trifluoropropan-2-yloxy)phenyl, 3-cyano-4-((R)-1,1,1-trifluoropropan-2-yloxy)phenyl, 3-halo-4-isopropylamino-phenyl, 3-cyano-4-isopropylamino-phenyl, 3-halo-4-((R)-1,1,1-trifluoropropan-2-ylamino)phenyl, and 3-cyano-4-((R)-1,1,1-trifluoropropan-2-ylamino)phenyl.

In some embodiments, X is —(CR₁₀R₁₁)ₘ and m is 0. In some embodiments, X is —(CR₁₀R₁₁)ₘ and m is 1. In some embodiments, X is —(CR₁₀R₁₁)ₘ and m is 2.

In some embodiments, X is —(CR₁₀R₁₁)ₙC(R₁₃)═C(R₁₄)— and n is 0.

In some embodiments, X is NR₈ and R₈ is hydrogen.

In some embodiments, X is —(CR₁₀R₁₁)ₘ, R₁₀ is hydrogen, R₁₁ is hydroxy, and m is 1. In some embodiments, X is —(CHOH)CH₂—.

In some embodiments, Y is —C(O)—. In some embodiments, Y is a direct bond linking X and Z.

In some embodiments, Z is NR₈.

In some embodiments, Z is —(CR₁₀R₁₁)_{q} and q is 0.

In some embodiments, Z is —O(CR₁₀R₁₁)ₛ and s is 0. In some embodiments, Z is —O(CR₁₀R₁₁)ₛ and s is 1. In some embodiments, Z is —O(CR₁₀R₁₁)ₛ and s is 2.

In some embodiments, —X—Y-Z- is chosen from —CH₂NR₈—, —CH₂C(O)NR₈—, —NR₈C(O)NR₈—, —CH₂CH₂C(O)NR₈—, —CH═CH—, —CH₂—, —CH₂CH₂—, —CH(OH)—CH₂—NR₈—, —NR₈C(O)—, —C(O)O—, —C(O)OCH₂CH₂—, —C(O)CH₂—, and —CH(OH)—CH₂—.

In some embodiments, R₂ is chosen from hydrogen, optionally substituted alkoxy, and optionally substituted amino. In some embodiments, R₂ is hydrogen.

In some embodiments, R₃ is chosen from hydrogen, aminocarbonyl, optionally substituted amino, optionally substituted lower alkyl, optionally substituted heterocycloalkyl, and optionally substituted heteroaryl. In some embodiments, R₃ is chosen from carbamoyl, (mono-lower alkyl)carbamoyl, optionally substituted amino, and optionally substituted lower alkyl.

In some embodiments, R₄ is chosen from optionally substituted lower alkyl and optionally substituted aryl. In some embodiments, R₄ is chosen from phenyl and benzyl, each of which is substituted with an optionally substituted heteroaryl group and each of which phenyl and benzyl is optionally further substituted with a group chosen from halo, hydroxy, and lower alkyl. In some embodiments, R₄ is chosen from phenyl and benzyl, each of which is substituted with a heteroaryl group chosen from
7,8-dihydro-imidazo[1,2-c][1,3]oxazin-2-yl,
3a,7a-dihydro-1H-benzoimidazol-2-yl,
imidazo[2,1-b]oxazol-6-yl,
oxazol-4-yl,
5,6,7,8-tetrahydro-imidazo[1,2-a]pyridin-2-yl,
1H-[1,2,4]triazol-3-yl,
2,3-dihydro-imidazol-4-yl,
1H-imidazol-2-yl,
imidazo[1,2-a]pyridin-2-yl,
thiazol-2-yl,
thiazol-4-yl,
pyrazol-3-yl, and
1H-imidazol-4-yl,
   each of which heteroaryl groups is optionally substituted with one, two, or three groups chosen from optionally substituted lower alkyl, halo, acyl, sulfonyl, cyano, nitro, optionally substituted amino, and optionally substituted heteroaryl.

Also provided is at least one chemical entity chosen from compounds of Formula II: 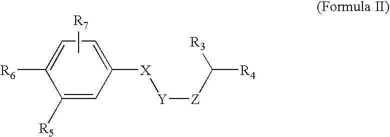
and pharmaceutically acceptable salts, solvates, chelates, non-covalent complexes, prodrugs, and mixtures thereof, wherein X, Y, Z, R₃, and R₄ are as described for compounds of Formula I and wherein
R₅ is chosen from optionally substituted heterocycloalkyl, optionally substituted lower alkyl, nitro, cyano, hydrogen, sulfonyl, and halo;
R₆ is chosen from hydrogen, halo, optionally substituted alkyl, optionally substituted cycloalkyl, optionally substituted heterocycloalkyl, optionally substituted amino, sulfanyl, optionally substituted alkoxy, cycloalkyloxy, optionally substituted heterocycloalkyloxy, optionally substituted aryloxy, and optionally substituted heteroaryloxy; and
R₇ is chosen from hydrogen, acyl, optionally substituted alkyl, optionally substituted cycloalkyl, optionally substituted alkoxy, halo, hydroxy, nitro, cyano, optionally substituted amino, sulfonyl, carboxyalkyl, aminocarbonyl, optionally substituted aryl, and optionally substituted heteroaryl.

In some embodiments, R₅ is hydrogen, cyano, nitro, or halo. In some embodiments, R₅ is chloro or cyano.

In some embodiments, R₆ is optionally substituted lower alkoxy, optionally substituted lower alkyl, or optionally substituted amino. In some embodiments, R₆ is lower alkoxy, 2,2,2-trifluoro-1-methyl-ethoxy, lower alkylamino or 2,2,2-trifluoro- 1-methyl-ethylamino. In some embodiments, R₆ is propoxy, 2,2,2-trifluoro-1-methyl-ethoxy, propylamino, or 2,2,2-trifluoro-1-methyl-ethylamino.

In some embodiments, R₇ is hydrogen.

Also provided is at least one chemical entity chosen from compounds of Formula III 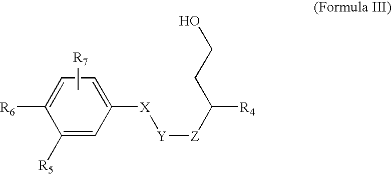
and pharmaceutically acceptable salts, solvates, chelates, non-covalent complexes, prodrugs, and mixtures thereof, wherein X, Y, Z, and R₄ are as described for compounds of Formula I and wherein R₅, R₆, and R₇ are as described for compounds of Formula II.

Also provided is at least one chemical entity chosen from compounds of Formula IV 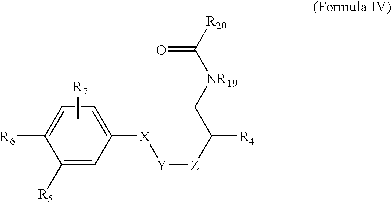
and pharmaceutically acceptable salts, solvates, chelates, non-covalent complexes, prodrugs, and mixtures thereof, wherein X, Y, Z, and R₄ are as described for compounds of Formula I, wherein R₅, R₆, and R₇ are as described for compounds of Formula II, and wherein
R₁₉ is chosen from hydrogen and optionally substituted lower alkyl; and
R₂₀ is chosen from optionally substituted alkyl, optionally substituted cycloalkyl, optionally substituted aryl, optionally substituted heterocycloalkyl, optionally substituted heteroaryl, optionally substituted alkoxy, and optionally substituted amino.

In some embodiments, R₁₉ is chosen from hydrogen and lower alkyl. In some embodiments, R₁₉ is hydrogen.

In some embodiments, R₂₀ is chosen from optionally substituted alkyl, optionally substituted amino, and optionally substituted heterocycloalkyl. In some embodiments, R₂₀ is chosen from (dimethylamino)methyl, azetidin-1-ylmethyl, pyrrolidin-1-ylmethyl, (methylamino)methyl, aminomethyl, amino, methylamino, and dimethylamino.

Also provided is at least one chemical entity chosen from compounds of Formula V: 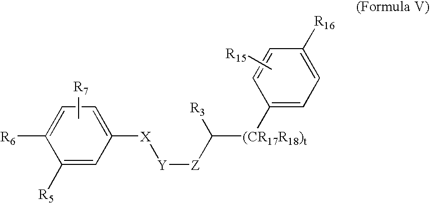
and pharmaceutically acceptable salts, solvates, chelates, non-covalent complexes, prodrugs, and mixtures thereof, wherein X, Y, Z, and R₃ are as described for compounds of Formula I, wherein R₅, R₆, and R₇ are as described for compounds of Formula II, and wherein
t is chosen from 0, 1, and 2;
R₁₇ and R₁₈ are independently chosen from hydrogen, hydroxy, optionally substituted alkyl, optionally substituted aryl, optionally substituted heterocycloalkyl, or optionally substituted cycloalkyl;
R₁₆ is optionally substituted heteroaryl; and
R₁₅ is chosen from hydrogen, halo, hydroxy, and lower alkyl.

In some embodiments, R₁₆ is chosen from
7,8-dihydro-imidazo[1,2-c][1,3]oxazin-2-yl,
3a,7a-dihydro-1H-benzoimidazol-2-yl,
imidazo[2,1-b]oxazol-6-yl,
oxazol-4-yl,
5,6,7,8-tetrahydro-imidazo[1,2-a]pyridin-2-yl,
1H-[1,2,4]triazol-3-yl,
2,3-dihydro-imidazol-4-yl,
1H-imidazol-2-yl,
imidazo[1,2-a]pyridin-2-yl,
thiazol-2-yl,
thiazol-4-yl,
pyrazol-3-yl, and
1H-imidazol-4-yl,
   each of which is optionally substituted with one, two, or three groups chosen from optionally substituted lower alkyl, halo, acyl, sulfonyl, cyano, nitro, optionally substituted amino, and optionally substituted heteroaryl.

In some embodiments, R₁₆ is chosen from
1H-imidazol-2-yl,
imidazo[1,2-a]pyridin-2-yl; and
1H-imidazol-4-yl,
   each of which is optionally substituted with one or two groups chosen from optionally substituted lower alkyl, halo, and acyl.

In some embodiments, R₁₅ is hydrogen.

In some embodiments, the compound of Formula I is chosen from
(3E)(2R)-4-[3-chloro-4-(methylethoxy)phenyl]-N-methyl-2-[(4-phenylphenyl)methyl]but-3-enamide;
2-amino-1-[3-chloro-4-(methylethoxy)phenyl]ethan-1-ol;
2-amino-N-[3-chloro-4-(methylethoxy)phenyl]-3-[4-(phenylmethoxy)phenyl]propanamide;
N-[3-chloro-4-(methylethoxy)phenyl]-2-[(methylamino)carbonylamino]-3-[4-(phenylmethoxy)phenyl]propanamide;
1-[3-chloro-4-(methylethoxy)phenyl]-2-({[4-(phenylmethoxy)phenyl]methyl}amino)ethan-1-ol;
1-[3-chloro-4-(methylethoxy)phenyl]-2-((2-hydroxyethyl){[4-(phenylmethoxy)phenyl]methyl}amino)ethan-1-ol;
2-{[(dimethylamino)sulfonyl]amino}-N-[3-chloro-4-(methylethoxy)phenyl]-3-[4-(phenylmethoxy)phenyl]propanamide;
(1S)-1-({4-[2-(tert-butyl)-1-methylimidazol-4-yl]phenyl}methyl)-3-hydroxypropyl 3-cyano-4-(methylethoxy)benzoate;
(1S)-1-({4-[2-(tert-butyl)-1-methylimidazol-4-yl]phenyl}methyl)-3-hydroxypropyl 3-chloro-4-(methylethoxy)benzoate;
(3S)-3-(acetylamino)-4-{4-[2-(tert-butyl)imidazol-4-yl]phenyl}butyl 3-cyano-4-(methylethoxy)benzoate;
(3S)-3-({[3-chloro-4-(methylethoxy)phenyl]methyl}amino)-4-{4-[2-(1-hydroxy-isopropyl)-1-methylimidazol-4-yl]phenyl}butan-1-ol;
(4E)(3S)-3-({4-[2-(tert-butyl)-1-methylimidazol-4-yl]phenyl}methyl)-5-[3-chloro-4-(methylethoxy)phenyl]pent-4-en-1-ol;
(3S)-4-{4-[2-(tert-butyl)-1-methylimidazol-4-yl]phenyl }-3-({[3-chloro-4-(methylethoxy)phenyl]methyl}amino)butanoic acid;
(3S)-3-({4-[2-(tert-butyl)-1-methylimidazol-4-yl]phenyl}methyl)-1-[3-chloro-4-(methylethoxy)phenyl]-5-hydroxypentan-1-one; and
(3S)-3-({4-[2-(tert-butyl)-1-methylimidazol-4-yl]phenyl}methyl)-1-[3-chloro-4-(methylethoxy)phenyl]pentane-1,5-diol.

The starting materials and other reactants are commercially available, e.g., from Aldrich Chemical Company, Milwaukee, Wis., or may be readily prepared by those skilled in the art using commonly employed synthetic methodology.

Unless specified otherwise, the terms “solvent”, “inert organic solvent” or “inert solvent” mean a solvent inert under the conditions of the reaction being described in conjunction therewith, including, for example, benzene, toluene, acetonitrile, tetrahydrofuran (“THF”), dimethylformamide (“DMF”), chloroform, methylene chloride (or dichloromethane), diethyl ether, methanol, pyridine and the like. Unless specified to the contrary, the solvents used in the reactions of the present invention are inert organic solvents.

In general, esters of carboxylic acids may be prepared by conventional esterification procedures, for example alkyl esters may be prepared by treating the required carboxylic acid with the appropriate alkanol, generally under acidic conditions. Likewise, amides may be prepared using conventional amidation procedures, for example amides may be prepared by treating an activated carboxylic acid with the appropriate amine. Alternatively, a lower-alkyl ester such as a methyl ester of the acid may be treated with an amine to provide the required amide, optionally in presence of trimethylalluminium following the procedure described in Tetrahedron Lett. 48, 4171-4173, (1977). Carboxyl groups may be protected as alkyl esters, for example methyl esters, which esters may be prepared and removed using conventional procedures, one convenient method for converting carbomethoxy to carboxyl is to use aqueous lithium hydroxide.

The salts and solvates mentioned herein may as required be produced by methods conventional in the art. For example, if an inventive compound is an acid, a desired base addition salt can be prepared by treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; such as ethylenediamine, and cyclic amines, such as cyclohexylamine, piperidine, morpholine, and piperazine; as well as inorganic salts derived from sodium, calcium, potassium, magnesium, manganese, iron, copper, zinc, aluminum, and lithium.

If a compound is a base, a desired acid addition salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, or the like.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can, of course, also be used.

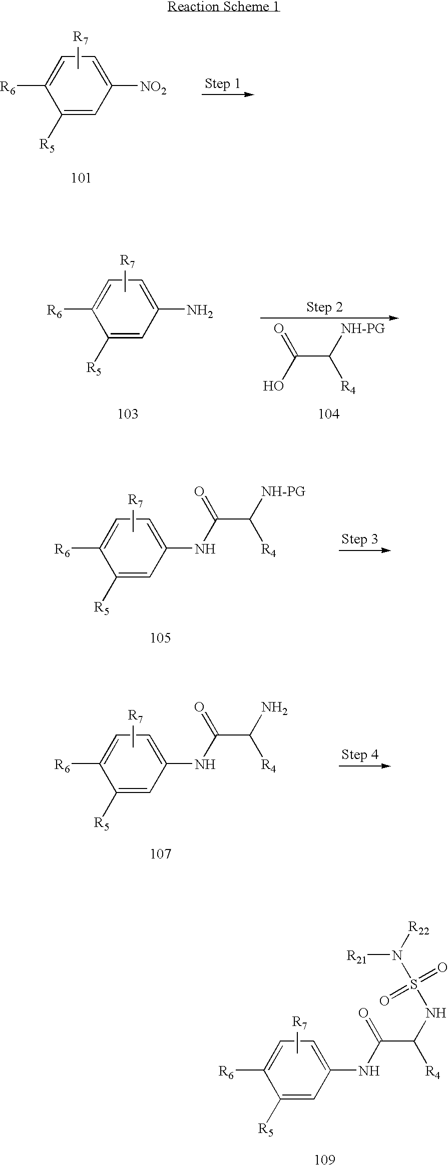

Referring to Reaction Scheme 1, Step 1, to a stirred mixture of a compound of Formula 101 and an excess (such as about 3 equivalents) of iron powder in a polar, protic solvent such as methanol is added concentrated HCl. After the reaction solution is stirred at room temperature for overnight, product, a compound of Formula 103, is isolated and optionally purified.

Referring to Reaction Scheme 1, Step 2, to a solution of an excess (such as about 1.2 equivalents) of a compound of Formula 104 wherein PG is a protecting group such as Boc in an inert solvent such as DMF is added an excess (such as about 1.2 equivalents) of HBTU and a compound of Formula 103. The reaction mixture is stirred for about 14 h. The product, a compound of Formula 105, is isolated and optionally purified.

Referring to Reaction Scheme 1, Step 3, the protecting group, PG, is removed to afford the free amine. For example, to a solution of a compound of Formula 105 in an inert solvent such as DCM is added TFA. The reaction mixture is stirred for about 4 h. The product, a compound of Formula 107, is isolated and optionally purified.

Referring to Reaction Scheme 1, Step 4, to a solution of a compound of Formula 107, such as the TFA salt, in an inert solvent such as DCM is added an excess (such as about 2 equivalents) of a compound of Formula Cl—S(O)₂R₂₁R₂₂ (wherein R₂₁ and R₂₂ are independently chosen from hydrogen and optionally substituted lower alkyl), a base such as DIEA, and DMAP. The reaction mixture is stirred for about 14 h. The product, a compound of Formula 109, is isolated and optionally purified.

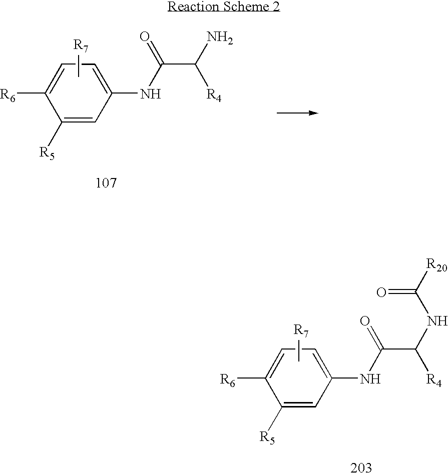

Referring to Reaction Scheme 2, to a solution of a compound of Formula 107, such as the TFA salt of a compound of Formula 107, in an inert solvent such as DCM is added an excess (such as about 2 equivalents) of an isocyanate of Formula R₂₀—CO wherein R₂₀ is optionally substituted amino and a base such as DIEA. The reaction mixture is stirred for about 4 h. The product, a compound of Formula 203, is isolated and optionally purified.

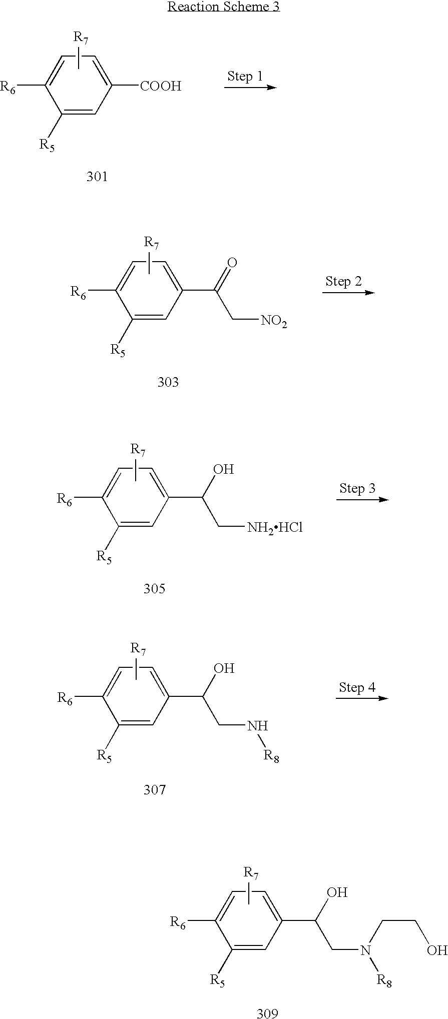

Referring to Reaction Scheme 3, Step 1, to a solution of a compound of Formula 301 in an inert solvent such as DMF is added about an equivalent of diethyl cyanophosphonate and a base such as triethylamine at about 0° C. The reaction mixture is allowed to warm to room temperature and stirred for about 14 h. The product, a compound of Formula 303, is isolated and optionally purified.

Referring to Reaction Scheme 3, Step 2, to a solution of a compound of Formula 303 in an inert solvent is added NaBH4 at about 0° C. The reaction mixture is stirred for about 90 min. LiAlH4 (such as 1 M LiAlH₄ in THF) is then added dropwise. The product, a compound of Formula 305, is isolated and optionally purified.

Referring to Reaction Scheme 3, Step 3, to a solution of a compound of Formula 305 in an inert solvent such as DCM is added a base such as DIEA, Na(OAc)₃BH, and an excess (such as 1.1 equivalents) of an aldehyde of Formula R_{8′}CHO wherein R_{8′} is chosen from optionally substituted alkyl, optionally substituted cycloalkyl, optionally substituted heterocycloalkyl, optionally substituted aryl, and optionally substituted heteroaryl at about 0° C. The reaction mixture is allowed to warm to room temperature and stirred for about 14 h. The product, a compound of Formula 307, is isolated and optionally purified.

Referring to Reaction Scheme 3, Step 4, to a solution of a compound of Formula 307 in an inert solvent such as DCM are added Na(OAc)₃BH and an excess (such as about 1.1 equivalents) of an aldehyde of Formula H(CO)—CH₂—OTBDMS at about 0° C. The reaction mixture is allowed to warm to room temperature and stirred for 10 h. The product, a compound of Formula 309, is isolated and optionally purified.

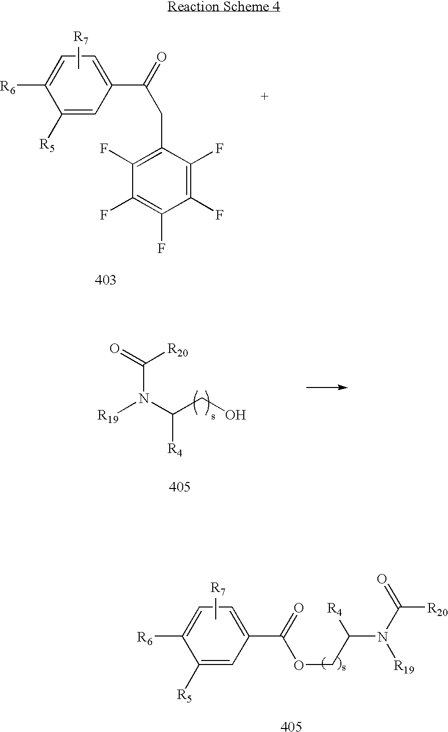

Referring to Reaction Scheme 4, to a solution of a compound of Formula 405 in an inert solvent such as DMF are added DIEA and an excess (such as about 1.2 equivalents) of a compound of Formula 403. The reaction mixture is stirred for about 1 hour. The product, a compound of Formula 405, is isolated and optionally purified.

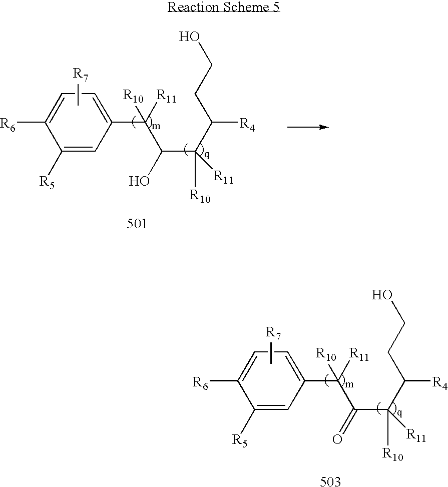

Referring to Reaction Scheme 5, to a stirred solution of a compound of Formula 501 in an inert solvent such as is added an excess of MnO₂. The reaction is refluxed for about 18 h. The product, a compound of Formula 503, is isolated and optionally purified.

Once made, the chemical entities of the invention find use in a variety of applications involving alteration of mitosis. As will be appreciated by those skilled in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In some embodiments, the chemical entities of the invention are used to inhibit mitotic spindle formation, thus causing prolonged cell cycle arrest in mitosis. By “inhibit” in this context is meant decreasing or interfering with mitotic spindle formation or causing mitotic spindle dysfunction. By “mitotic spindle formation” herein is meant organization of microtubules into bipolar structures by mitotic kinesins. By “mitotic spindle dysfunction” herein is meant mitotic arrest.

The chemical entities of the invention bind to, and/or inhibit the activity of, one or more mitotic kinesin. In some embodiments, the mitotic kinesin is human, although the chemical entities may be used to bind to or inhibit the activity of mitotic kinesins from other organisms. In this context, “inhibit” means either increasing or decreasing spindle pole separation, causing malformation, i.e., splaying, of mitotic spindle poles, or otherwise causing morphological perturbation of the mitotic spindle. Also included within the definition of a mitotic kinesin for these purposes are variants and/or fragments of such protein and more particularly, the motor domain of such protein.

The chemical entities of the invention are used to treat cellular proliferation diseases. Such disease states which can be treated by the chemical entities provided herein include, but are not limited to, cancer (further discussed below), autoimmune disease, fungal disorders, arthritis, graft rejection, inflammatory bowel disease, cellular proliferation induced after medical procedures, including, but not limited to, surgery, angioplasty, and the like. Treatment includes inhibiting cellular proliferation. It is appreciated that in some cases the cells may not be in an abnormal state and still require treatment. Thus, in some embodiments, the invention herein includes application to cells or individuals afflicted or subject to impending affliction with any one of these disorders or states.

The chemical entities, pharmaceutical formulations and methods provided herein are particularly deemed useful for the treatment of cancer including solid tumors such as skin, breast, brain, cervical carcinomas, testicular carcinomas, etc. More particularly, cancers that can be treated include, but are not limited to:
Cardiac: sarcoma (angiosarcoma, fibrosarcoma, rhabdomyosarcoma, liposarcoma), myxoma, rhabdomyoma, fibroma, lipoma and teratoma;
Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver: hepatoma (hepatocellular carcinoma), cholangiocarcinoma, hepatoblastoma, angiosarcoma, hepatocellular adenoma, hemangioma;
Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma);
Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma);
Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma];
Skin: malignant melanoma, basal cell carcinoma, squamous cell carcinoma, Karposi's sarcoma, moles dysplastic nevi, lipoma, angioma, dermatofibroma, keloids, psoriasis; and
Adrenal glands: neuroblastoma.
   As used herein, treatment of cancer includes treatment of cancerous cells, including cells afflicted by any one of the above-identified conditions. Thus, the term “cancerous cell” as provided herein, includes a cell afflicted by any one of the above identified conditions.

Another useful aspect of the invention is a kit having at least one chemical entity described herein and a package insert or other labeling including directions treating a cellular proliferative disease by administering an effective amount of the at least one chemical entity. The chemical entity in the kits of the invention is particularly provided as one or more doses for a course of treatment for a cellular proliferative disease, each dose being a pharmaceutical formulation including a pharmaceutical excipient and at least one chemical entity described herein.

For assay of mitotic kinesin-modulating activity, generally either a mitotic kinesin or at least one chemical entity described herein is non-diffusably bound to an insoluble support having isolated sample receiving areas (e.g., a microtiter plate, an array, etc.). The insoluble support may be made of any composition to which the sample can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the sample is not crucial so long as it is compatible with the reagents and overall methods of the invention, maintains the activity of the sample and is nondiffusable. Particular methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the sample, excess unbound material is removed by washing. The sample receiving areas may then be blocked through incubation with bovine serum albumin (BSA), casein or other innocuous protein or other moiety.

The chemical entities of the invention may be used on their own to inhibit the activity of a mitotic kinesin. In some embodiments, at least one chemical entity of the invention is combined with a mitotic kinesin and the activity of the mitotic kinesin is assayed. Kinesin activity is known in the art and includes one or more of the following: the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes, such as kinases or proteases; and specific kinesin cellular activities such as spindle pole separation.

Methods of performing motility assays are well known to those of skill in the art. (See e.g., Hall, et al. (1996), Biophys. J., 71: 3467-3476, Turner et al., 1996, AnaL Biochem. 242 (1):20-5; Gittes et al., 1996, Biophys. J. 70(1): 418-29; Shirakawa et al., 1995, J. Exp. BioL 198: 1809-15; Winkelmann et al., 1995, Biophys. J. 68: 2444-53; Winkelmann et al., 1995, Biophys. J. 68: 72S.)

Methods known in the art for determining ATPase hydrolysis activity also can be used. Suitably, solution based assays are utilized. U.S. Pat. No. 6,410,254, hereby incorporated by reference in its entirety, describes such assays. Alternatively, conventional methods are used. For example, Pᵢ release from kinesin (and more particularly, the motor domain of a mitotic kinesin) can be quantified. In some embodiments, the ATPase hydrolysis activity assay utilizes 0.3 M PCA (perchloric acid) and malachite green reagent (8.27 mM sodium molybdate II, 0.33 mM malachite green oxalate, and 0.8 mM Triton X-100). To perform the assay, 10 μL of the reaction mixture is quenched in 90 μL of cold 0.3 M PCA. Phosphate standards are used so data can be converted to mM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to mM Pᵢ and plotted over time. Additionally, ATPase assays known in the art include the luciferase assay.

ATPase activity of kinesin motor domains also can be used to monitor the effects of agents and are well known to those skilled in the art. In some embodiments ATPase assays of kinesin are performed in the absence of microtubules. In some embodiments, the ATPase assays are performed in the presence of microtubules. Different types of agents can be detected in the above assays. In some embodiments, the effect of an agent is independent of the concentration of microtubules and ATP. In some embodiments, the effect of the agents on kinesin ATPase can be decreased by increasing the concentrations of ATP, microtubules or both. In some embodiments, the effect of the agent is increased by increasing concentrations of ATP, microtubules or both.

Chemical entities that inhibit the biochemical activity of a mitotic kinesin in vitro may then be screened in vivo. In vivo screening methods include assays of cell cycle distribution, cell viability, or the presence, morphology, activity, distribution, or number of mitotic spindles. Methods for monitoring cell cycle distribution of a cell population, for example, by flow cytometry, are well known to those skilled in the art, as are methods for determining cell viability. See for example, U.S. Pat. No. 6,437,115, hereby incorporated by reference in its entirety. Microscopic methods for monitoring spindle formation and malformation are well known to those of skill in the art (see, e.g., Whitehead and Rattner (1998), J. Cell Sci. 111:2551-61; Galgio et al, (1996) J. Cell Biol., 135:399-414), each incorporated herein by reference in its entirety.

The chemical entities of the invention inhibit one or more mitotic kinesins. One measure of inhibition is IC₅₀, defined as the concentration of the chemical entity at which the activity of the mitotic kinesin is decreased by fifty percent relative to a control. In some embodiments, the at least one chemical entity has an IC₅₀ of less than about 1 mM. In some embodiments, the at least one chemical entity has an IC₅₀ of less than about 100 μM. In some embodiments, the at least one chemical entity has an IC₅₀ of less than about 10 μM. In some embodiments, the at least one chemical entity has an IC₅₀ of less than about 1 μM. In some embodiments, the at least one chemical entity has an IC₅₀ of less than about 100 nM. In some embodiments, the at least one chemical entity has an IC₅₀ of less than about 10 nM. Measurement of IC₅₀ is done using an ATPase assay such as described herein.

Another measure of inhibition is Kᵢ. For chemical entities with IC₅₀'s less than 1 μM, the Kᵢ or K_{d} is defined as the dissociation rate constant for the interaction of the compounds described herein with a mitotic kinesin. In some embodiments, the at least one chemical entity has a Kᵢ of less than about 100 μM. In some embodiments, the at least one chemical entity has a Kᵢ of less than about 10 μM. In some embodiments, the at least one chemical entity has a Kᵢ of less than about 1 μM. In some embodiments, the at least one chemical entity has a Kᵢ of less than about 100 nM. In some embodiments, the at least one chemical entity has a Kᵢ of less than about 10 nM.

The Kᵢ for a chemical entity is determined from the IC₅₀ based on three assumptions and the Michaelis-Menten equation. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ I - \frac{( {E}_{0} + {I}_{0} + Kd ) - \sqrt{{( {E}_{0} + {I}_{0} + Kd )}^{2} - 4 {E}_{0} {I}_{0}}}{2 {E}_{0}} ]$$

where V is the observed rate, Vₘₐₓ is the rate of the free enzyme, I₀ is the inhibitor concentration, E₀ is the enzyme concentration, and K_{d} is the dissociation constant of the enzyme-inhibitor complex.

Another measure of inhibition is GI₅₀, defined as the concentration of the chemical entity that results in a decrease in the rate of cell growth by fifty percent. In some embodiments, the at least one chemical entity has a GI₅₀ of less than about 1 mM. In some embodiments, the at least one chemical entity has a GI₅₀ of less than about 20 μM. In some embodiments, the at least one chemical entity has a GI₅₀ of less than about 10 μM. In some embodiments, the at least one chemical entity has a GI₅₀ of less than about 1 μM. In some embodiments, the at least one chemical entity has a GI₅₀ of less than about 100 nM. In some embodiments, the at least one chemical entity has a GI₅₀ of less than about 10 nM. Measurement of GI₅₀ is done using a cell proliferation assay such as described herein. Chemical entities of this class were found to inhibit cell proliferation.

In vitro potency of small molecule inhibitors is determined, for example, by assaying human ovarian cancer cells (SKOV3) for viability following a 72-hour exposure to a 9-point dilution series of compound. Cell viability is determined by measuring the absorbance of formnazon, a product formed by the bioreduction of MTS/PMS, a commercially available reagent. Each point on the dose-response curve is calculated as a percent of untreated control cells at 72 hours minus background absorption (complete cell kill).

The chemical entities described herein inhibit CENP-E. One measure of inhibition is IC₅₀, defined as the concentration of the chemical entity at which the activity of CENP-E is decreased by fifty percent relative to a control. In some embodiments, the at least one chemical entity has a IC₅₀ of less than about 1 mM. In some embodiments, the at least one chemical entity has a IC₅₀ of less than about 100 μM. In some embodiments, the at least one chemical entity has a IC₅₀ of less than about 10 μM. In some embodiments, the at least one chemical entity has a IC₅₀ of less than about 1 μM. In some embodiments, the at least one chemical entity has a IC₅₀ of less than about 100 nM. In some embodiments, the at least one chemical entity has a IC₅₀ of less than about 10 nM. Measurement of IC₅₀ is done using an ATPase assay such as described herein.

Anti-proliferative compounds that have been successfully applied in the clinic to treatment of cancer (cancer chemotherapeutics) have GI₅₀'S that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation, irrespective of the concentration demonstrating inhibition, have potential clinical usefulness.

To employ the chemical entities of the invention in a method of screening for compounds that bind to a mitotic kinesin, the mitotic kinesin is bound to a support, and a compound of the invention is added to the assay. Alternatively, the chemical entity of the invention is bound to the support and a mitotic kinesin is added. Classes of compounds among which novel binding agents may be sought include specific antibodies, non-natural binding agents identified in screens of chemical libraries, peptide analogs, etc. Of particular interest are screening assays for candidate agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the chemical entities of the invention to a mitotic kinesin may be done in a number of ways. In some embodiments, the chemical entity is labeled, for example, with a fluorescent or radioactive moiety, and binding is determined directly. For example, this may be done by attaching all or a portion of a mitotic kinesin to a solid support, adding a labeled test compound (for example a chemical entity of the invention in which at least one atom has been replaced by a detectable isotope), washing off excess reagent, and determining whether the amount of the label is that present on the solid support.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the kinesin proteins may be labeled at tyrosine positions using ¹²⁵I, or with fluorophores. Alternatively, more than one component may be labeled with different labels; using ¹²⁵I for the proteins, for example, and a fluorophor for the antimitotic agents.

The chemical entities of the invention may also be used as competitors to screen for additional drug candidates. “Candidate agent” or “drug candidate” or grammatical equivalents as used herein describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. Screens of this sort may be performed either in the presence or absence of microtubules. In the case where protein binding or activity is screened, particular embodiments exclude molecules already known to bind to that particular protein, for example, polymer structures such as microtubules, and energy sources such as ATP. Particular embodiments of assays herein include candidate agents which do not bind the cellular proliferation protein in its endogenous native state termed herein as “exogenous” agents. In some embodiments, exogenous agents further exclude antibodies to the mitotic kinesin.

Candidate agents can encompass numerous chemical classes, though typically they are small organic compounds having a molecular weight of more than 100 and less than about 2,500 daltons. Candidate agents comprise functional groups necessary for structural interaction with proteins, particularly hydrogen bonding and lipophilic binding, and typically include at least an amine, carbonyl, hydroxy, ether, or carboxyl group, generally at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, and/or amidification to produce structural analogs.

Competitive screening assays may be done by combining a mitotic kinesin and a drug candidate in a first sample. A second sample comprises at least one chemical entity of the present invention, a mitotic kinesin and a drug candidate. This may be performed in either the presence or absence of microtubules. The binding of the drug candidate is determined for both samples, and a change, or difference in binding between the two samples indicates the presence of a drug candidate capable of binding to a mitotic kinesin and potentially inhibiting its activity. That is, if the binding of the drug candidate is different in the second sample relative to the first sample, the drug candidate is capable of binding to a mitotic kinesin.

In some embodiments, the binding of the candidate agent to a mitotic kinesin is determined through the use of competitive binding assays. In some embodiments, the competitor is a binding moiety known to bind to the mitotic kinesin, such as an antibody, peptide, binding partner, ligand, etc. Under certain circumstances, there may be competitive binding as between the candidate agent and the binding moiety, with the binding moiety displacing the candidate agent.

In some embodiments, the candidate agent is labeled. Either the candidate agent, or the competitor, or both, is added first to the mitotic kinesin for a time sufficient to allow binding, if present. Incubations may be performed at any temperature which facilitates optimal activity, typically between 4 and 40° C.

Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In some embodiments, the competitor is added first, followed by the candidate agent. Displacement of the competitor is an indication the candidate agent is binding to the mitotic kinesin and thus is capable of binding to, and potentially inhibiting, the activity of the mitotic kinesin. In some embodiments, either component can be labeled. Thus, for example, if the competitor is labeled, the presence of label in the wash solution indicates displacement by the agent. Alternatively, if the candidate agent is labeled, the presence of the label on the support indicates displacement.

In some embodiments, the candidate agent is added first, with incubation and washing, followed by the competitor. The absence of binding by the competitor may indicate the candidate agent is bound to the mitotic kinesin with a higher affinity. Thus, if the candidate agent is labeled, the presence of the label on the support, coupled with a lack of competitor binding, may indicate the candidate agent is capable of binding to the mitotic kinesin.

Inhibition is tested by screening for candidate agents capable of inhibiting the activity of a mitotic kinesin comprising the steps of combining a candidate agent with a mitotic kinesin as above, and determining an alteration in the biological activity of the mitotic kinesin. Thus, in some embodiments, the candidate agent should both bind to the mitotic kinesin (although this may not be necessary), and alter its biological or biochemical activity as defined herein. The methods include both in vitro screening methods and in vivo screening of cells for alterations in cell cycle distribution, cell viability, or for the presence, morpohology, activity, distribution, or amount of mitotic spindles, as are generally outlined above.

Alternatively, differential screening may be used to identify drug candidates that bind to the native mitotic kinesin but cannot bind to a modified mitotic kinesin.

Positive controls and negative controls may be used in the assays. Suitably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Accordingly, the chemical entities of the invention are administered to cells. By “administered” herein is meant administration of a therapeutically effective dose of at least one chemical entity of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant any cell in which mitosis or meiosis can be altered.

A “patient” for the purposes of the present invention includes both humans and other animals, particularly mammals, and other organisms. Thus the methods are applicable to both human therapy and veterinary applications. In some embodiments, the patient is a mammal, and more particularly, the patient is human.

Chemical entities of the invention having the desired pharmacological activity may be administered, in some embodiments, as a pharmaceutically acceptable composition comprising an pharmaceutical excipient, to a patient, as described herein. Depending upon the manner of introduction, the chemical entities may be formulated in a variety of ways as discussed below. The concentration of the at least one chemical entity in the formulation may vary from about 0.1-100 wt. %.

The agents may be administered alone or in combination with other treatments, i.e., radiation, or other chemotherapeutic agents such as the taxane class of agents that appear to act on microtubule formation or the camptothecin class of topoisomerase I inhibitors. When used, other chemotherapeutic agents may be administered before, concurrently, or after administration of at least one chemical entity of the present invention. In one aspect of the invention, at least one chemical entity of the present invention is co-administered with one or more other chemotherapeutic agents. By “co-administer” it is meant that the at least one chemical entity is administered to a patient such that the at least one chemical entity as well as the co-administered compound may be found in the patient's bloodstream at the same time, regardless when the compounds are actually administered, including simultaneously.

The administration of the chemical entities of the present invention can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the compound or composition may be directly applied as a solution or spray.

Pharmaceutical dosage forms include at least one chemical entity described herein and one or more pharmaceutical excipients. As is known in the art, pharmaceutical excipients are secondary ingredients which function to enable or enhance the delivery of a drug or medicine in a variety of dosage forms (e.g.: oral forms such as tablets, capsules, and liquids; topical forms such as dermal, opthalmic, and otic forms; suppositories; injectables; respiratory forms and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance, to ease handling and administration of dose, for convenience of use, or to control bioavailability. While pharmaceutical excipients are commonly described as being inert or inactive, it is appreciated in the art that there is a relationship between the properties of the pharmaceutical excipients and the dosage forms containing them.

Pharmaceutical excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995), each of which is incorporated herein by reference for all purposes.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colors, flavors, sweetening agents, polymers, waxes or other solubility-retarding materials.

Compositions for intravenous administration will generally comprise intravenous fluids, i.e., sterile solutions of simple chemicals such as sugars, amino acids or electrolytes, which can be easily carried by the circulatory system and assimilated. Such fluids are prepared with water for injection USP.

Dosage forms for parenteral administration will generally comprise fluids, particularly intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, The Science and Practice of Pharmacy [full citation previously provided], and include:
alcohol, e.g., 5% alcohol (e.g., in dextrose and water (“D/W”) or D/W in normal saline solution (“NSS”), including in 5% dextrose and water (“D5/W”), or D5/W in NSS);
synthetic amino acid such as Aminosyn, FreAmine, Travasol, e.g., 3.5 or 7; 8.5; 3.5, 5.5 or 8.5 % respectively;
ammonium chloride e.g., 2.14%;
dextran 40, in NSS e.g., 10% or in D5/W e.g., 10%;
dextran 70, in NSS e.g., 6% or in D5/W e.g., 6%;
dextrose (glucose, D5/W) e.g., 2.5-50%;
dextrose and sodium chloride e.g., 5-20% dextrose and 0.22-0.9% NaCl;
lactated Ringer's (Hartmann's) e.g., NaCl 0.6%, KCl 0.03%, CaCl₂ 0.02%;
lactate 0.3%;
mannitol e.g., 5%, optionally in combination with dextrose e.g., 10% or NaCl e.g., 15 or 20%;
multiple electrolyte solutions with varying combinations of electrolytes, dextrose, fructose, invert sugar Ringer's e.g., NaCl 0.86%, KCl 0.03%, CaCl₂ 0.033%;
sodium bicarbonate e.g., 5%;
sodium chloride e.g., 0.45, 0.9, 3, or 5%;
sodium lactate e.g., 1/6 M; and
sterile water for injection
   The pH of such IV fluids may vary, and will typically be from 3.5 to 8 as known in the art.

The chemical entityies of the invention can be administered alone or in combination with other treatments, i.e., radiation, or other therapeutic agents, such as the taxane class of agents that appear to act on microtubule formation or the camptothecin class of topoisomerase I inhibitors. When so-used, other therapeutic agents can be administered before, concurrently (whether in separate dosage forms or in a combined dosage form), or after administration of an active agent of the present invention.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All publications, including but not limited to patents and patent applications, cited in this specification are herein incorporated by reference as if each individual publication were specifically and individually indicated to be incorporated by reference herein as though fully set forth.

### EXAMPLES

### Example 1

### Preparation of 3-(4-(benzyloxy)phenyl)-N-(3-chloro-4-isopropoxyphenyl)-2-(N,N-dimethylsulfamoylamino)propanamide

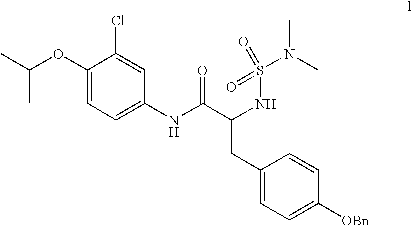 Experimental Section:

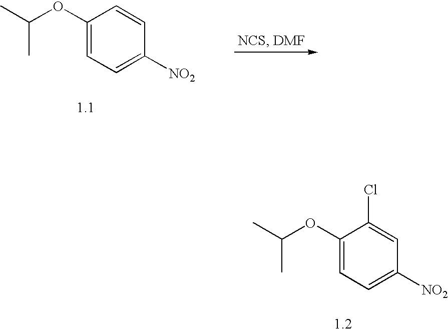

To a solution of 1-isopropoxy-4-nitrobenzene 1.1 (2 g, 11.0 mmol) in DMF (15 mL) was added N-chlorosuccinimide (NCS) (1.8 g, 13.2 mmol) at 0° C. After stirring at 0° C. for 4 h, the reaction mixture was allowed to warm to room temperature and stirred for 14 h. The mixture was purified on RP-HPLC using a mixture of acetonitrile and H₂O to give 1.2 (1.8 g, 76%), which was characterized by ¹H NMR.

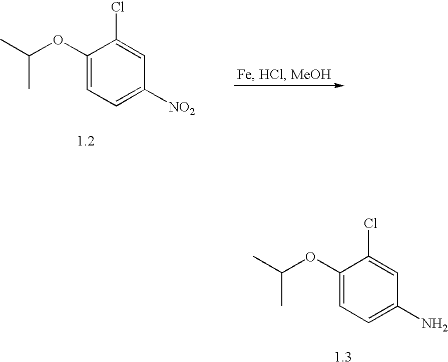

To a stirred mixture of 1.2 (1.7 g, 7.88 mmol), iron powder (1.3 g, 23.2 mmol) and methanol (35 mL) was added concentrated HCl (4 mL). After the reaction solution was stirred at room temperature overnight, the pH of the solution was basified by adding saturated sodium NaHCO₃ and diluted with DCM (250 mL). The organic layer was washed with H₂O and brine, dried over sodium sulfate, filtered, and concentrated to give 1.3, which was used without further purification in the next step.

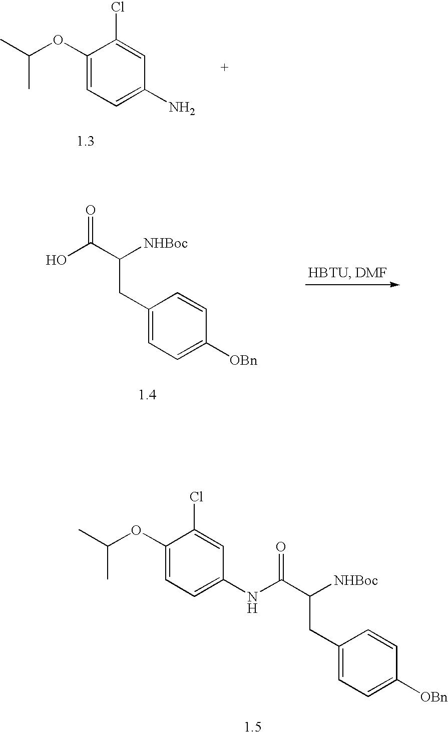

To a solution of 1.4 (1.2 g, 3.2 mmol) in DMF (10 mL) were added HBTU (1.18 g, 3.2 mmol) and crude 1.3 (480 mg, 2.6 mmol). The reaction mixture was stirred for 14 h. The mixture was purified on RP-HPLC using a mixture of acetonitrile and H₂O to give 1.5 (450 mg, 32% from 1.2). LRMS (M+H⁺) m/z 539.1.

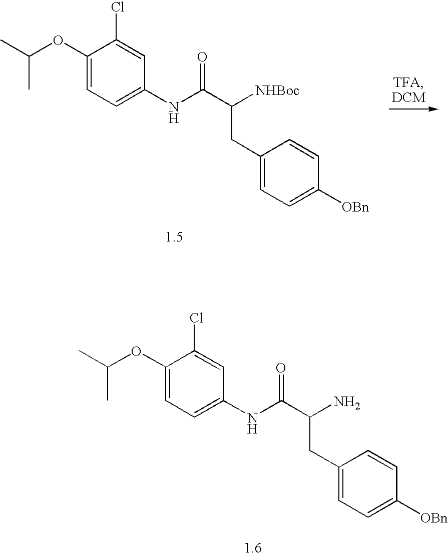

To a solution of 1.5 (400 mg, 0.74 mmol) in DCM (20 mL) was added TFA (2 mL). The reaction mixture was stirred for 4 h. The mixture was concentrated and purified on RP-HPLC using a mixture of acetonitrile and H₂O to give 1.6 as a TFA salt (400 mg, quant.). LRMS (M+H⁺) m/z 439.0.

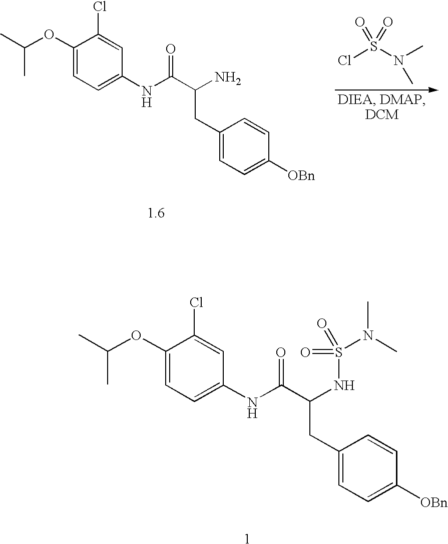

To a solution of 1.6 (TFA salt, 50 mg, 0.09 mmol) in DCM (5 mL) were added dimethylsulfamoyl chloride (32 uL, 0.18 mmol), DIEA (79 uL, 0.45 mmol) and DMAP (20 mg, 0.18 mmol). The reaction mixture was stirred for 14 h. The mixture was concentrated and purified on RP-HPLC using a mixture of acetonitrile and H₂O to give 1 (15 mg, 31%). LRMS (M+H⁺) m/z 546.5.

### Example 2

### Preparation of 3-(4-(benzyloxy)phenyl)-N-(3-chloro-4-isopropoxyphenyl)-2-(3-methylureido)propanamide

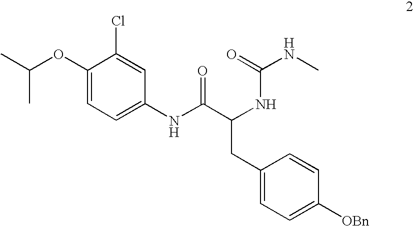 Experimental Section:

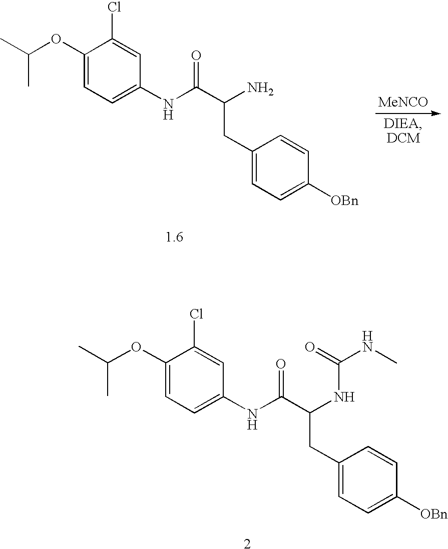

To a solution of 6 (TFA salt, 50 mg, 0.09 mmol) in DCM (5 mL) were added MeNCO (10 uL, 0.18 mmol) and DIEA (32 uL, 0.18 mmol). The reaction mixture was stirred for 4 h. The mixture was concentrated and purified on RP-HPLC using a mixture of acetonile and H₂O to give 8 (45 mg, quant.). LRMS (M+H⁺) m/z 496.1.

### Example 3

### Preparation of 2-((4-(benzyloxy)benzyl)(2-hydroxyethyl)amino)-1-(3-chloro-4-isopropoxyphenyl)ethanol

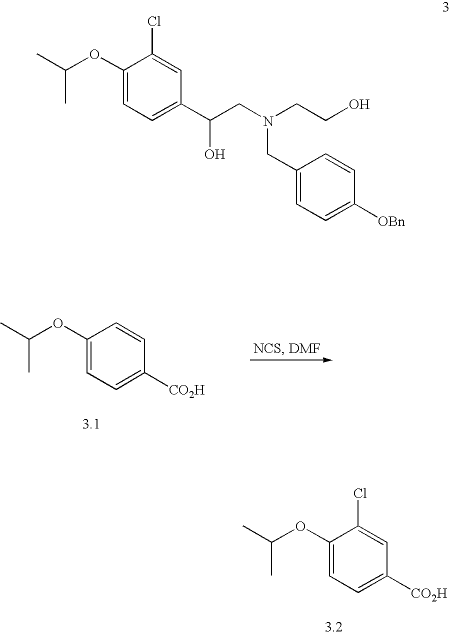

To a solution of 4-isopropoxylbenzoic acid 3.1 (25 g, 140 mmol) in DMF (150 mL) was added NCS (24 g, 182 mmol). The reaction mixture was stirred overnight. H₂O (500 mL) was added to the reaction mixture. The precipitate was collected, washed with water and dried to give 3.2 (26.4 g, 88%) as a white solid, which was used in the next step without further purification.

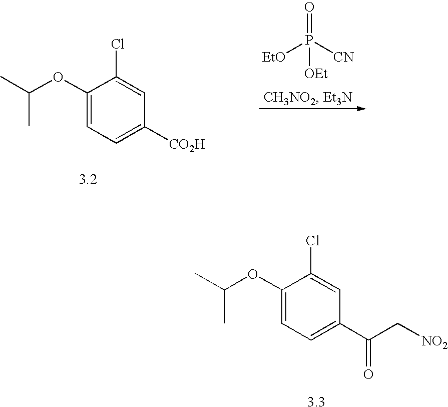

To a solution of 3.2 (10 g, 46.6 mmol) and CH₃NO₂ (20.95 mL, 39 mmol) in DMF (40 mL) were added diethyl cyanophosphonate (7.1 mL, 46.6 mmol) and triethylamine (16.4 mL, 117 mmol) at 0° C. The reaction mixture was allowed to warm to room temperature and stirred for 14 h. The mixture was concentrated to give 3.3, which was used without further purification in the next step.

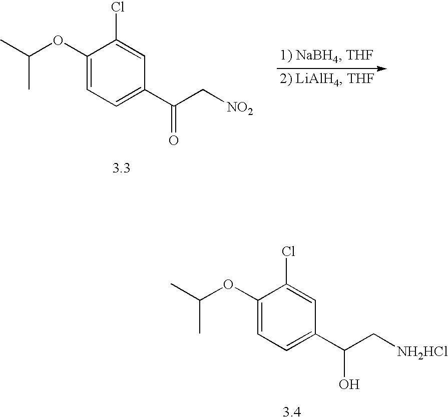

To a solution of 3.3 (1.3 g, 5 mmol) in THF (35 mL) was added NaBH₄ (230 mg, 6 mmol) at 0° C. The reaction mixture was stirred for 90 min. LiAlH₄ (1 M in THF, 6 mL) was then added dropwise. The reaction mixture was quenched with MeOH (7 mL) and concentrated. The resulting residue was extracted with EtOAc. The EtOAc solution was washed with HCl (1M). The aqueous layer was then concentrated and dried under high vacuum. The resulting sold was extracted with MeOH, filtered, concentrated, and purified on RP-HPLC using a mixture of acetonitrile and H₂O to give 3.4 (105 mg, 9% from 3.2). LRMS (M+H⁺—H₂O) m/z 212.0.

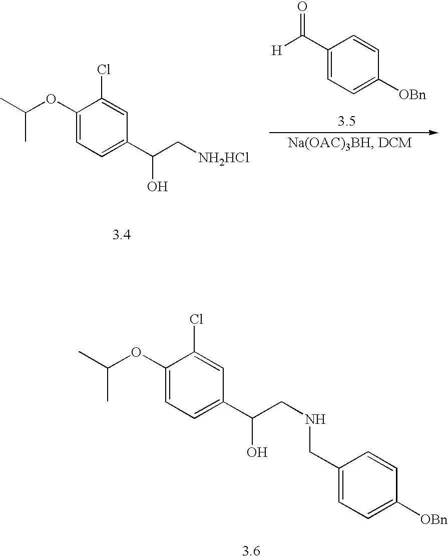

To a solution of 3.4 (250 mg, 0.94 mmol) in DCM (6 mL) were added DIEA (99 uL, 0.56 mmol), Na(OAc)₃BH (260 mg, 1.23 mmol) and aldehyde 3.5 (220 mg, 1.03 mmol) at 0° C. The reaction mixture was allowed to warm to room temperature and stirred for 14 h. The reaction mixture was quenched with saturated NaHCO₃ and diluted with DCM. The organic layer was washed with brine, dried over Na₂SO₄, concentrated, and purified on RP-HPLC using a mixture of acetonitrile and H₂O to give 3.6 (120 mg, 30%). LRMS (M+H⁺) m/z 426.1.

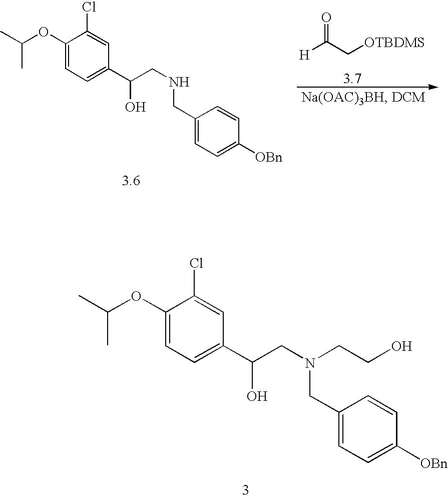

To a solution of 3.6 (60 mg, 0.14 mmol) in DCM (5 mL) were added Na(OAc)₃BH (36 mg, 0.17 mmol) and aldehyde 3.7 (30 mg, 0.17 mmol) at 0° C. The reaction mixture was allowed to warm to room temperature and stirred for 10 h. The reaction mixture was quenched with saturated NaHCO₃ and diluted with DCM. The organic layer was washed with brine, dried over Na₂SO₄, concentrated, and purified on RP-HPLC using a mixture of acetonitrile and H₂O to give 3 (5 mg, 8%). LRMS (M+H⁺) m/z 470.5.

### Example 4

### Preparation of (S)-3-acetamido-4-(4-(2-tert-butyl-1H-imidazol-4-yl)phenyl)butyl 3-cyano-4-isopropoxybenzoate

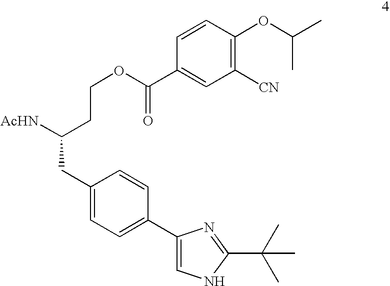 Experimental Section:

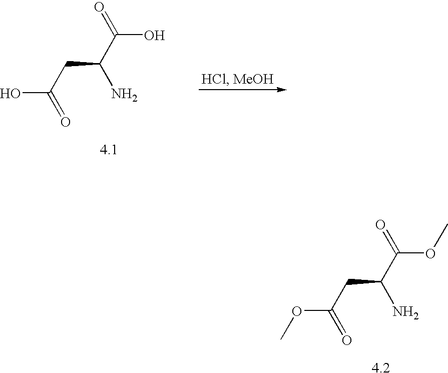

To a solution of D-aspartic acid 4.1 (59 g, 0.376 mol) in methanol (200 mL) was bubbled HCl gas at 0° C. for 10 minutes. The reaction mixture was allowed to warm to room temperature and stir overnight. The mixture was concentrated, and the resulting residue was dried to give 4.2 as a HCl salt (0.376 mol) which was used without further purification. LRMS (M−H⁺) m/z 162.0.

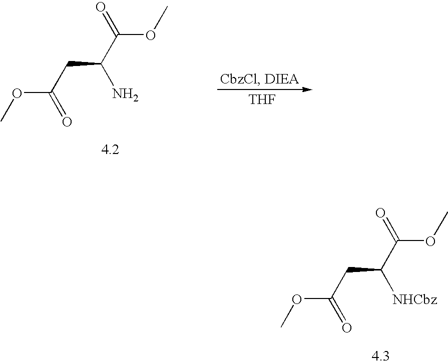

To a stirred solution of 4.2 (0.376 mol) and DIEA (196 mL, 1.13 mol) in THF (200 mL) was added benzyl chloroformate (59.0 mL, 0.414 mol) dropwise. After the reaction solution was stirred at room temperature for 1 hr, the solution was concentrated. The resulting residue was dissolved in NaHCO₃ solution (300 mL) and extracted with DCM (100 mL ×3). The combined DCM solution was dried over sodium sulfate, filtered, and concentrated to give 4.3 (0.376 mol) which was used without further purification. LRMS (M+H⁺) m/z 296.1.

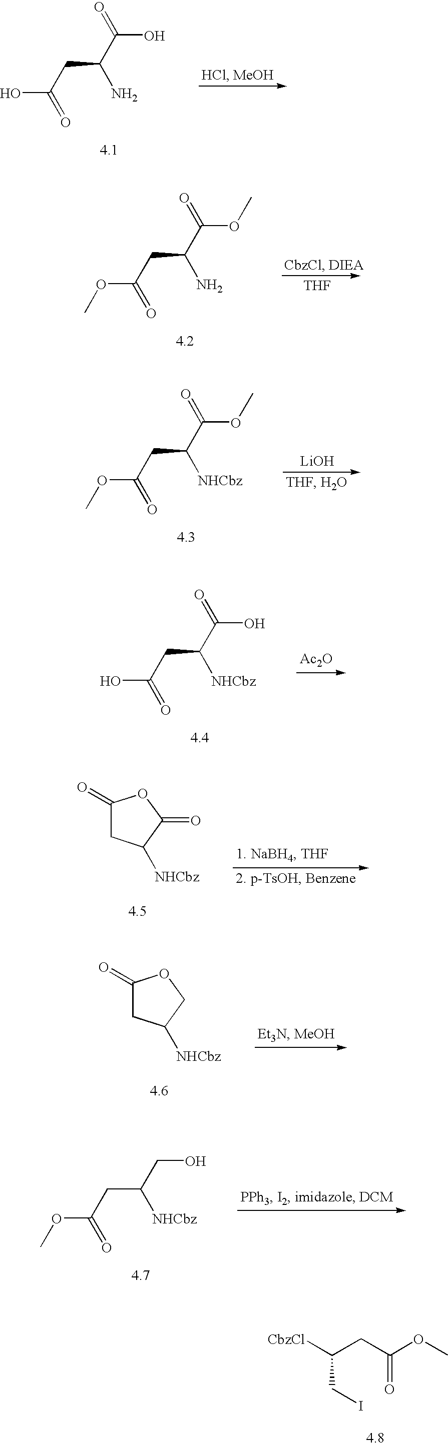

To a solution of 4.3 (0.376 mol) in THF (200 mL) and H₂O (100 mL) was added lithium hydroxide (31.6 g, 0.752 mol). The reaction mixture was stirred for 2 hours. The reaction mixture was filtered through a silica gel plug (the pH of the filtrate was about 7) and concentrated. The residue was dried to give 4.4 (0.376 mol) which was used without further purification. LRMS (M+H⁺) m/z 268.1.

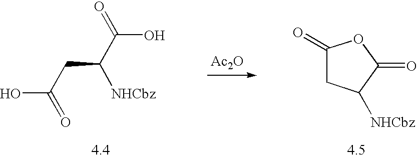

A solution of 4.4 (0.376 mol) in acetic anhydride (200 mL) was stirred for 1 hour. The reaction mixture was concentrated, and the residue was dried under vacuum to give 4.5 (0.376 mol) which was used without further purification.

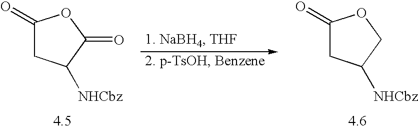

To a solution of 4.5 (0.376 mol) in THF (1000 mL) was added sodium borohydride (14.2 g, 0.376 mol) at 0° C. over 30 minutes. The reaction mixture was stirred for 3 hours. The mixture was acidified to pH˜2 using HCl (4N). The solution was concentrated to about one quarter, diluted with water (300 mL) and extracted by ethyl ether (200 mL×3). The combined ether solution was dried over sodium sulfate, filtered, and concentrated in vacuum. The resulting residue was dissolved in benzene (300 mL) to which TsOH (500 mg) was added. The reaction mixture was stirred at reflux 3 hrs. The solution was concentrated to about 100 mL and ether (200 mL) was added to form precipitate. The precipitate was filtered, washed with ether and dried to give 4.6 (57.5 g, 65% from 4.1). LRMS (M+H⁺) m/z 236.1.

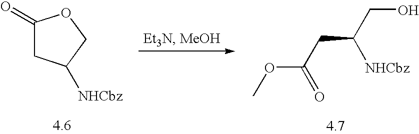

A solution of 4.6 (30.0 g, 0.128 mol) in methanol (200 mL) and triethylamine (142 mL, 1.02 mol) was stirred overnight. The reaction mixture was concentrated and the residue dried to give 4.7, which was directly used in the next step. LRMS (M+H⁺) m/z 268.1.

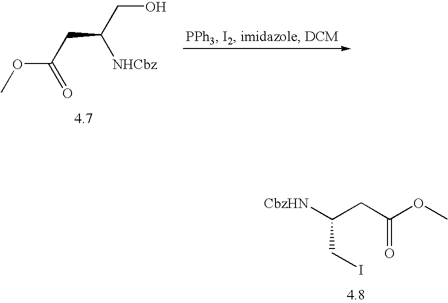

A solution of compound 4.7 (0.128 mol), Ph₃P (50.4 g, 0.192 mol) and imidazole (13.1 g, 0.192 mol) in DCM (300 mL) was stirred at 0° C. for 10 min. Iodine (48.7 g, 0.192 mol) was added in portions over 15 minutes. The reaction mixture was allowed to warm to room temperature and stirred for 1 hour. The solid was filtered away. The filtrate was washed with saturated Na₂SO₃ (200 mL×2) and brine (200 mL). The organic layer was dried over sodium sulfate, filtered, and concentrated. The resulting residue was purified on silica gel (hexanes:EtOAc 4:1 to 1:1) to give compound 4.8 (27.5 g, 59.2% from 4.6) as a white solid. LRMS (M+H⁺) m/z 378.0.

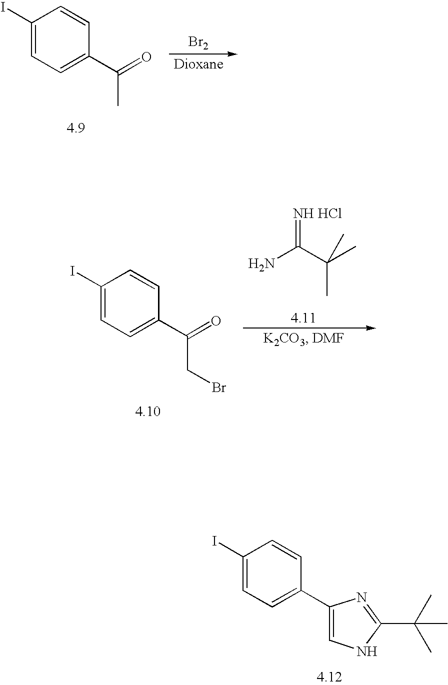

To a solution of 4-iodoacetophenone 4.9 (10 g, 40.6 mmol) in dioxane (100 mL) was added bromine (2.18 mL, 42.7 mmol) dropwise at 0° C. The reaction mixture was stirred for 30 minutes. The resulting solution was concentrated. The residue was dissolved in dichloromethane (200 mL) and was washed with satd. NaHCO₃, H₂O and brine, dried over Na₂SO₄, and concentrated to give 4.10. To a solution of 4.10 in DMF (100 mL) were added potassium carbonate (16.8 g, 122 mmol) and tert-butylcarbamidine hydrochloride 4.11 (11.1 g, 81.2 mmol). After stirring overnight, the reaction mixture was filtered and the filtrate was concentrated. The resulting residue was purified on silica gel using a mixture of hexanes and ethyl acetate to give 4.12 (8.1 g, 61%). LRMS (M+Na⁺) m/z 327.0.

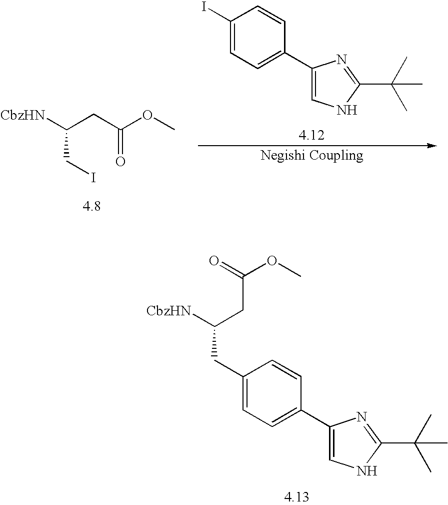

To a mixture of zinc powder (607 mg, 9.28 mmol) and DMF (5 mL) purged with nitrogen for 10 minutes was added 1,2-dibromoethane (45.8 uL, 0.532 mmol). The mixture was heated with a heat gun for ˜2 minutes, allowed to cool for 5 minutes, heated with a heat gun again, and then cooled to room temperature. TMSCl (17 uL, 0.133 mmol) was added to the mixture. After the mixture was stirred for 30 minutes, 4.8 (500 mg, 1.33 mmol) was added. After 1 hour, LCMS showed complete consumption of 4.8. To the above reaction solution was added aryl iodide 4.12 (438 mg, 1.33 mmol), Pd₂(dba)₃ (30.4 mg, 0.0333 mmol) and tri-o-tolylphosphine (40.5 mg, 0.133 mmol). The reaction mixture was maintained at 50° C. for 1 hour (monitored by LC-MS analysis). The reaction mixture was directly loaded to a silica gel plug and eluted with hexanes:EtOAc (3:1 to 1:1) to give compound 4.13 (500 mg, 83%). LRMS (M+H⁺) m/z 450.2.

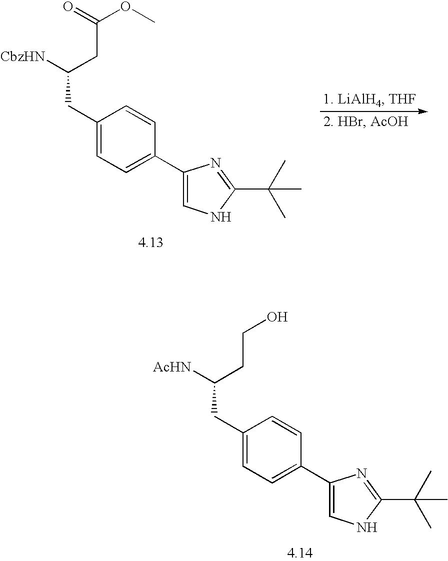

To a solution of 4.13 (500 mg, 1.11 mmol) in THF (50 mL) was added LiAlH₄ (2.22 mL, 2.22 mmol) dropwise at 0° C. The reaction mixture was stirred for 20 minutes. The solution was filtered through a silica gel plug and the filtrate was concentrated. The resulting residue was dissolved in concentrated hydrogen bromide in acetic acid (3 mL), stirred for 10 minutes, and concentrated. The residue was dissolved in ethyl acetate (100 mL) and washed with saturated NaHCO₃, H₂O and brine, dried over Na₂SO₄, and concentrated to give 4.14 (250 mg), which was used without further purification. LRMS (M+H⁺) m/z 330.2.

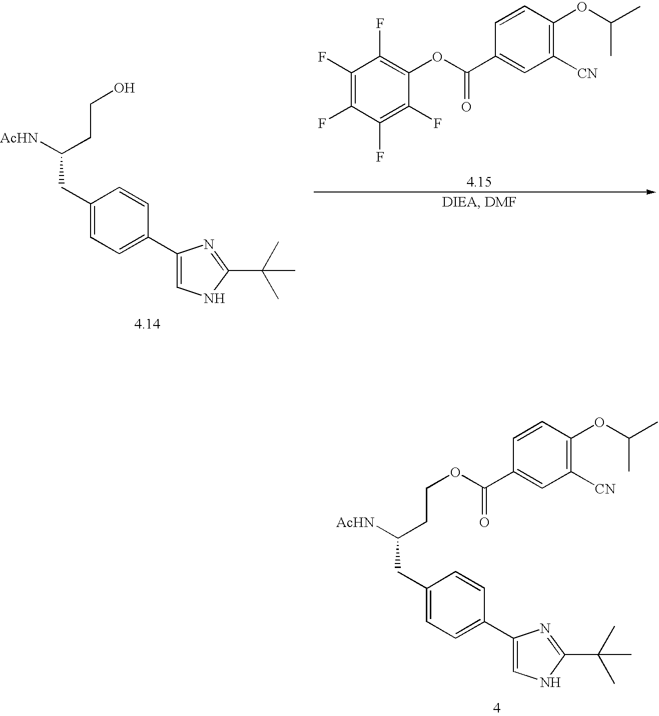

To a solution of 4.14 (250 mg, 0.759 mmol) in DMF (2 mL) were added DIEA (159 uL, 0.911 mmol) and 4.15 (338 mg, 0.911 mmol). The reaction mixture was stirred for 1 hour and then concentrated. The resulting residue was purified on silica gel using a mixture of hexanes and ethyl acetate to give 4 (180 mg, 45%). LRMS (M+H⁺) m/z 517.3.

### Example 5

### Preparation of [(3S,4E)-3-[4-(2-t-butyl-1-methyl-1H-imidazol-4-yl)benzyl]-5-(3-chloro-4-isopropoxyphenyl)-4-penten-1-ol

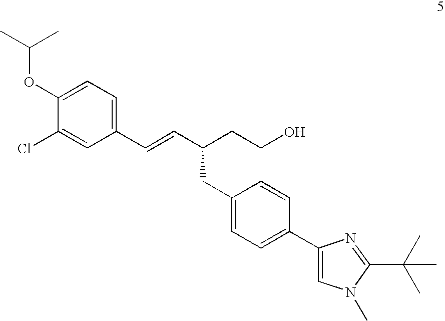 Experimental Section:

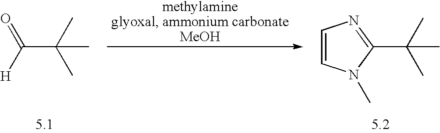

### [(3S,4E)-3-(4-bromobenzyl)-5-(3-chloro-4-isopropoxyphenyl)-4-penten-1-ol 2-t-butyl-1-methyl-1H-imidazole (5.2)

To a stirred solution of pivaldehyde (40 g, 464 mMol) at 0° C. was added a solution of 2 M methylamine in methanol (250 mL, 500 mMol). After stirring for 2 h, ammonium carbonate (23 g, 240 mMol) and trimeric glyoxal dihydrate (33 g, 157 mMol) was added. The reaction was slowly allowed to warm to room temperature and stirred for 18 h. (The reaction started out as a suspension then became a clear brown solution.) The reaction was concentrated under vacuum and distilled by short path under high vacuum. The title product (28.65 g, 44%) was obtained as a pale yellow oil that distilled over at 64-66° C., 0.2 mm Hg: ¹H NMR (400 MHz, CDCl₃) [symbol] 6.88 (d, J=1.2 Hz, 1H), 6.77 (d, J=1.2 Hz, 1H), 3.77 (s, 3 H), 1.45 (s, 9 H).

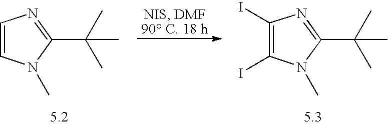

### 2-t-butyl-4,5-diiodo-1-methyl-1H-imidazole (5.3)

To a stirred solution of 2-t-butyl-1-methyl-1H-imidazole (28.36 g, 205 mMol) in DMF (400 mL) was added portionwise N-iodosuccinimide (113 g, 502 mMol). The reaction was stirred and heated at 90° C. for 18 h under N₂. After cooling to RT the reaction was evaporated to dryness under vacuum. The remaining residue was taken up in EtOAc, washed with aq. Sodium thiosulfate, brine, dried (Na₂SO₄), filtered and evaporated under vacuum. The solid was slurried in MeOH (100 mL). Water (100 mL) was slowly added with stirring to produce a white suspension that was filtered, rinsed with a small volume of (1:1) MeOH, H₂O, and dried under vacuum to produce the title compound (67.21 g, 84%) as a white solid: MS (ES) m/e 390.6 (M+H)⁺; ¹H NMR (400 MHz, CDCl₃) [symbol] 3.80 (s, 3 H), 1.44 (s, 9 H).

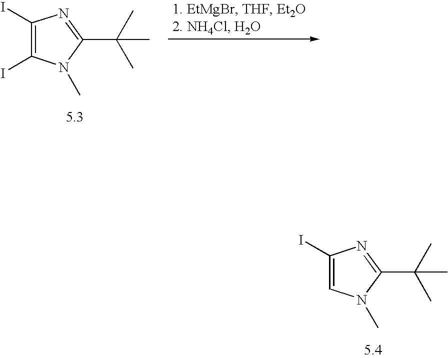

### 2-t-butyl-4-iodo-1-methyl-1H-imidazole (5.4)

To a stirred solution of 2-t-butyl-4,5-diiodo-1-methyl-1H-imidazole (25.19 g, 65 mMol) in THF (100 mL), under N₂ at 0° C., was added dropwise a solution of 3.0 M EtMgBr in Et₂O (25 mL, 75 mMol). (The reaction formed a cloudy suspension.) The reaction was stirred at 0° C. for 30 minutes then carefully quenched with sat. aq. NH₄Cl (50 mL). The title compound (16.58 g, 96%) was obtained as a pale yellow solid after extraction with EtOAc, washing with brine, drying with (Na₂SO₄), filtration and evaporation to dryness under vacuum: MS (ES) m/e 264.8 (M+H)⁺; ¹H NMR (400 MHz, CDCl₃) [symbol] 6.86 (s, 1 H), 3.75 (s, 3 H), 1.44 (s, 9 H).

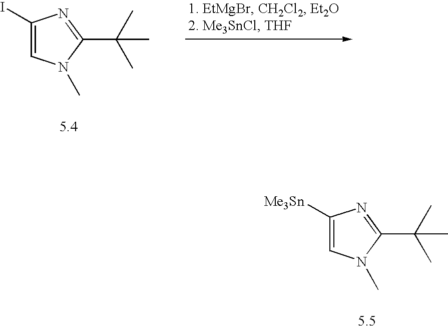

### 2-t-butyl-1-methyl-4-(trimethylstannanyl)-1H-imidazole (5.5)

To a stirred solution of 2-t-butyl-4-iodo-1-methyl-1H-imidazole (8.0 g, 30.3 mMol) in CH₂Cl₂ (80 mL) was added dropwise at RT under N₂ a solution of 3.0 M EtMgBr in Et₂O (12 mL, 36 mMol). After stirring for 1h, a solution of 1.0 N trimethyltin chloride in THF (32 mL, 32 mMol) was added dropwise over 5 minutes. The reaction was stirred at RT for 16 h, concentrated under vacuum, taken up in EtOAc, washed with aq. NH₄Cl, dried (MgSO₄), filtered and evaporated under vacuum. Short path distillation under vacuum (0.2 mmHg, 110-120° C.) gave the title compound (7.49 g, 82%) as a clear oil which solidified to a waxy solid in the refrigerator: 1H NMR (400 MHz, CDCl₃) [symbol] 6.81 (s, 1 H), 3.78 (s, 3 H), 1.48 (s, 9 H), 0.32 (s, 9 H).

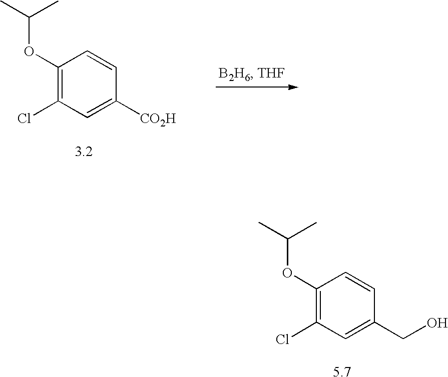

### 3-chloro-4-isopropoxybenzyl alcohol (5.7)

To a stirred solution of 3-chloro-4-isopropoxybenzoic acid (5.0 g, 23.3 mMol) in THF (50 mL) ar 0° C. under N₂ was added dropwise a solution of 1 N borane in THF (34 mL, 34 mMol) over 10 minutes. The reaction was allowed to warm to RT and stirred for 4 h. The reaction was recooled to 0° C. and slowly quenched with H₂O (10 mL). The reaction was concentrated under vacuum, taken up in EtOAc, washed with 1 N Na₂CO₃, brine, dried (MgSO₄), filtered and evaporated under vacuum to obtain the title compound (4.71 g, 100%) as a clear oil: ¹H NMR (400 MHz, CDCl₃) □7.40 (d, J=2.4 Hz, 1 H), 7.20 (dd, J=2.4, 8.4 Hz, 1H), 6.95 (d, J=8.4Hz, 1 H), 4.62 (s, 2 H), 4.56 (m, 1 H), 1.39 (d, J=6.4 Hz, 6 H).

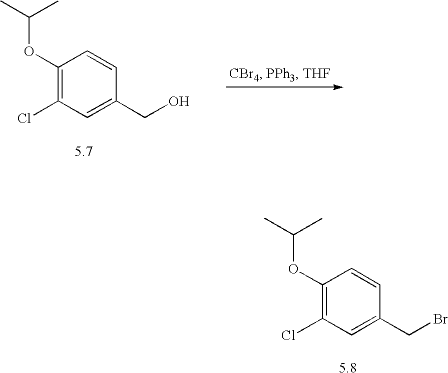

### 3-chloro-4-isopropoxybenzyl bromide (5.8)

To a stirred solution of 3-chloro-4-isopropoxybenzyl alcohol (4.0 g, 20 mMol) in THF (50 mL) was added with stirring at 0° C. carbon tetrabromide (8.0 g, 24 mMol) and triphenylphosphine (5.6 g, 21 mMol). The reaction was allowed to warm to RT and stirred for 18 h. The reaction was concentrated under vacuum, taken up in 5% EtOAc, hexane, filtered to remove the insolubles and purified by flash silica gel (5% EtOAc, hexane) to obtain the title compound (3.51 g, 66%) as a clear oil: 1H NMR (400 MHz, CDCl₃) □7.43 (d, J=2.4 Hz, 1 H), 7.24 (dd, J=2.4, 8.4 Hz, 1 H), 6.91 (d, J=8.4 Hz, 1 H), 4.58 (m, 1 H), 4.46 (s, 2 H), 1.40 (d, J=6.4 Hz, 6 H).

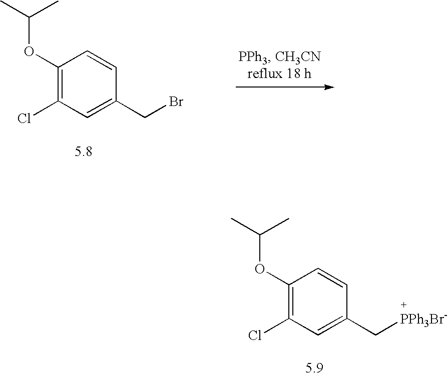

### 3-chloro-4-isopropoxy-benzyl(triphenyl)phosphonium bromide (5.9)

To a stirred solution of 3-chloro-4-isopropoxybenzyl bromide (3.5 g, 13.3 mMol) in acetonitrile (50 mL) was added triphenylphosphine (3.5 g, 13.3 mMol). The reaction was refluxed for 4 h, cooled to RT and evaporated to dryness under vacuum to give the title compound (6.98 g, 100%) as a white solid: MS (ES) m/e 445.2 (M−Br⁻)^{+.}

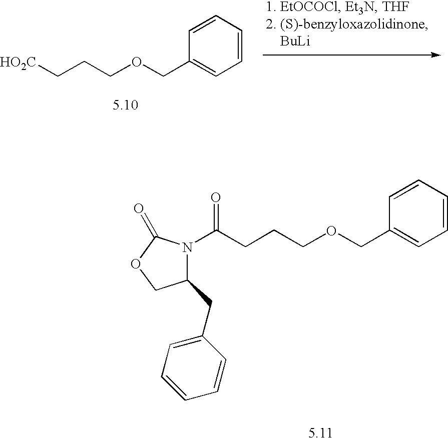

### (4S)-4-benzyl-3-(4-benzyloxybutanoyl)-1,3-oxazolidin-2-one (5.11)

To a stirred solution of 4-benzyloxybutanoic acid (5.1 g, 26.3 mMol) and Et₃N (3.8 mL, 27.2 mMol) in THF (200 mL) at 0° C. was added dropwise ethyl chloroformate (2.6 mL, 27.2 mMol). (A thick white slurry formed.) The reaction was allowed to warm to RT and stirred for 1 h.

In a separate flask a stirred solution of (4S)-4-benzyl-1,3-oxazolidin-2-one (4.66 g, 26.3 mMol) in THF (100 mL) was treated dropwise, at −78° C. under N₂, with a solution of 2.5 M BuLi in hexane (10.6 mL, 26.5 mMol). After stirring for 30 minutes the solution was cannulated over to the stirred mixed anhydride above at −78° C. After stirring for 30 minutes the reaction was allowed to warm to 0° C. and stirred for an additional 30 minutes. The reaction was quenched with sat. aq. NH₄Cl, concentrated under vacuum, taken up in EtOAc, washed with H₂O, brine, dried (MgSO₄), filtered and evaporated to dryness under vacuum. The title compound (7.00 g, 75%) was obtained by flash chromatography on silica gel (25% EtOAc, hexane) as a clear oil: MS (ES) m/e 354.2 (M+H)⁺.

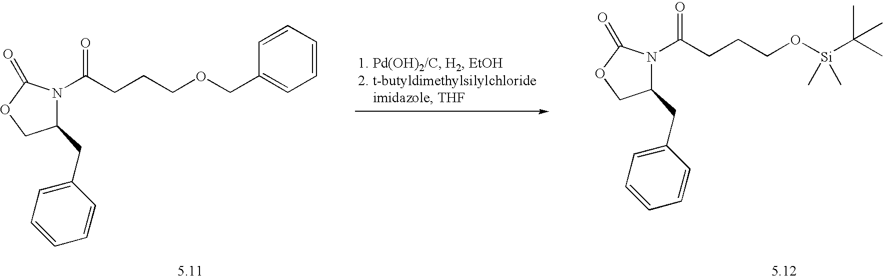

### (4S)-3-[4-(t-butyldimethylsilyloxy)butanoyl]-4-benzyl-1,3-oxazolidin-2-one (5.12)

To a stirred solution of (4S)-4-benzyl-3-(4-benzyloxybutanoyl)-1,3-oxazolidin-2-one (6.98 g, 19.8 mMol) in EtOH (150 mL) was added 20% Pd(OH)₂ on carbon (1 g). A balloon of H₂ was attached and the reaction stirred for 24 h. The reaction was filtered through a pad of Celite®, rinsed with EtOH and evaporated to dryness under vacuum. To the resulting clear oil and imidazole (3.0 g, 44 mMol) in THF (100 mL), with stirring at 0° C., was added dropwise a solution of t-butyldimethylsilyl chloride (6.0 g, 21.8 mMol) in THF (50 mL). (A thick white precipitate formed.) The reaction was allowed to warm to RT and stirred for 4 h. After concentration under vacuum the residue was taken up in EtOAc, washed with cold 0.5 N HCl, brine, dried (MgSO₄), filtered and evaporated under vacuum. The title compound (7.34 g, 98%) was obtained by flash chromatography on silica gel (20% EtOAc, hexane) as a clear oil: MS (ES) m/e 378.2 (M+H)⁺.

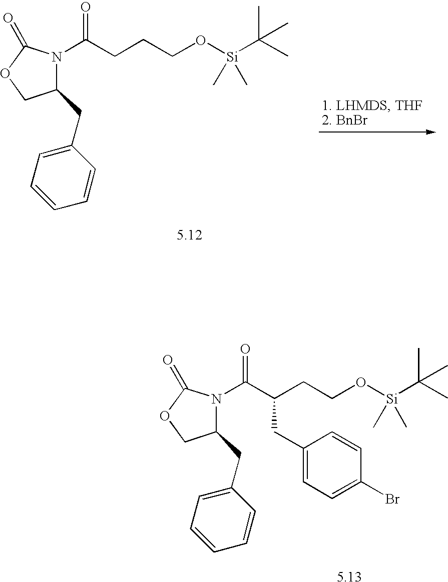

### (4S)-3-[(2S)-2-(4-bromobenzyl)-4-(t-butyldimethylsilyloxy)butanoyl]-4benzyl-1,3-oxazolidin-2-one (5.13)

To a stirred solution of (4S)-3-[4-(t-butyldimethylsilyloxy)butanoyl]-4-benzyl-1,3-oxazolidin-2-one (7.34 g, 19.4 mMol) in THF (100 mL) at −78° C. was added dropwise a solution of 1 N lithium bis(trimethylsilyl)amide in THF (27 mL, 27 mMol). After stirring at −78° C. for 1 h a solution of 4-bromobenzyl bromide (6.8 g, 27.2 mMol) in THF (10 mL) was added. The reaction was stirred for 1 h at −78° C., allowed to warm to 0° C., stirred for 1 h, and quenched with sat. aq. NH₄Cl. The reaction was extracted with EtOAc, washed with brine, dried (MgSO₄), filtered and evaporated under vacuum. Purification by flash chromatography on silica gel (10% EtOAc, hexane) gave the title compound (9.4 g, 88%) as a clear oil: MS (ES) m/e 546.2 (M+H)⁺.

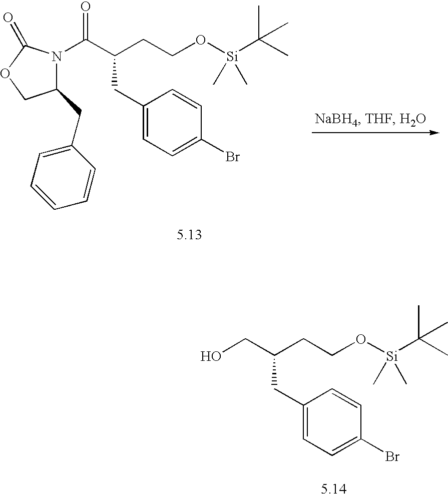

### (2S)-2-(4-bromobenzyl)-4-(t-butyldimethylsilyloxy)-1-butanol (5.14)

To a stirred solution of (4S)-3-[(2S)-2-(4-bromobenzyl)-4-(t-butyldimethylsilyloxy)butanoyl]-4-benzyl-1,3-oxazolidin-2-one (9.4 g, 17.2 mMol) in THF (150 mL) was added dropwise a solution of sodium borohydride (2.7 g, 71.4 mMol) in H₂O (30 mL). (The temperature was maintained at 20-25° C. Gas evolution was seen.) The reaction was stirred for 18 h, diluted with H₂O, extracted with EtOAc, washed with brine, dried (MgSO₄), filtered and evaporated under vacuum. Purification by flash chromatography on silica gel (15 to 20% EtOAc, hexane) gave the title compound (5.5 g, 85%) as a clear oil: MS (ES) m/e 373.2 (M+H)⁺.

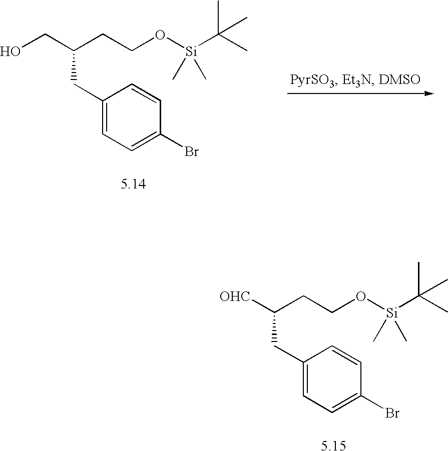

### (2S)-2-(4-bromobenzyl)-4-(t-butyldimethylsilyloxy)-1-butanal (5.15)

To a stirred solution of (2S)-2-(4-bromobenzyl)-4-(t-butyldimethylsilyloxy)-1-butanol (5.0 g, 13.4 mMol) and Et₃N (9.4 mL, 67 mMol) in CH₂Cl₂ (20 mL) was added a cloudy solution of sulfer trioxide pyridine complex (10.8 g, 67.8 mMol) in 20 mL DMSO. The reaction was stirred for 1 h at RT, diluted with CH₂Cl₂, washed with cold H₂O, dried (MgSO₄), filtered and evaporated under vacuum. Purification by flash chromatography on silica gel (5% EtOAc, hexane) gave the title compound (3.32 g, 67%) as a clear oil: MS (ES) m/e 371.2 (M+H)⁺.

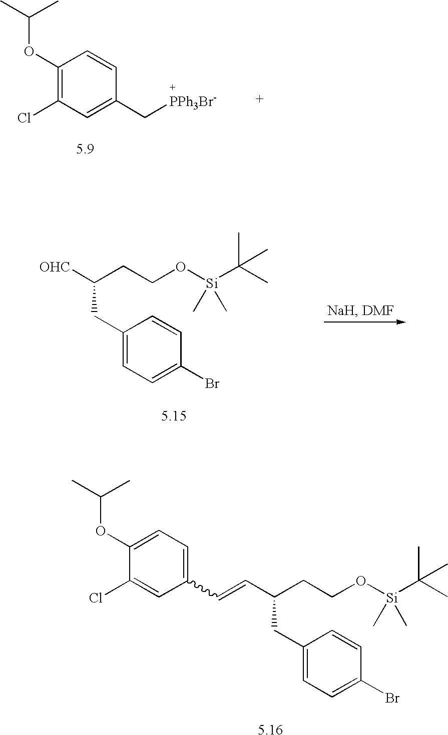

### [(3S,4E,Z)-3-(4-bromobenzyl)-5-(3-chloro-4-isopropoxyphenyl)-4-penten-1-yl]oxy]t-butyldimethylsilane (5.17)

To a stirred solution of 3-chloro-4-isopropoxy-benzyl(triphenyl)phosphonium bromide (6.98 g, 13.3 mMol) and (2S)-2-(4-bromobenzyl)-4-(t-butyldimethylsilyloxy)-1-butanal (4.9 g, 13.2 mMol) in DMF (50 mL) was added portionwise NaH 60% dispersion (0.53 g, 13.3 mMol) over 5 minutes. The reaction was stirred for 4 h at RT and evaporated to dryness under vacuum. The residue was taken up in EtOAc, washed with H₂O, brine, dried (MgSO₄), filtered and evaporated under vacuum. Purification by flash chromatography on silica gel (5% EtOAc, hexane) (loaded with CH₂Cl₂) gave the title compound (7.05 g, 98%) as a (2.3:1) mixture of trans and cis olefins: MS (ES) m/e 537.2 (M+H)⁺; ¹H NMR (400 MHz, CDCl₃) (Trans olefins) [symbol] 6.12 (d, J=16.0 Hz, 1 H), 5.85 (dd, J=16.0, 8.4 Hz, 1 H); (Cis olefins) [symbol] 6.31 (d, J=11.6 Hz, 1 H), 5.33 (app. t, J=11.6 Hz, 1 H).

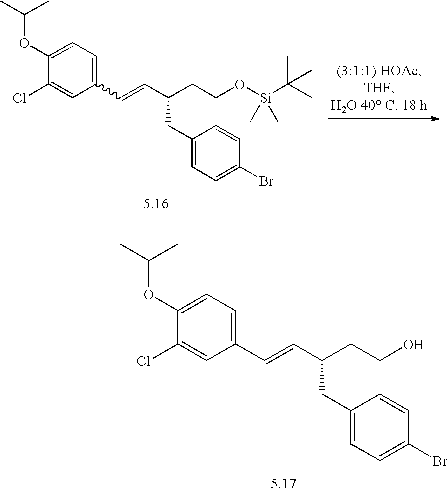

To [(3S,4E,Z)-3-(4-bromobenzyl)-5-(3-chloro-4-isopropoxyphenyl)-4-penten-1-yl]oxy]t-butyldimethylsilane (1.5 g, 2.8 mMol) was added a solution of (3:1:1) HOAc, THF, H₂O (20 mL). The reaction was stirred at 40° C. for 18 h, evaporated to dryness under vacuum and purified by flash chromatography on silica gel (25% EtOAc, hexanes) to give the title product (trans olefin isomer) (0.56 g, 48%) as a clear oil: MS (ES) m/e 555.2 (M+H)⁺.

An earlier fraction containing the cis olefin isomer (0.21 g, 18%) as well as a mixed olefin fraction (0.15 g, 13%) was also isolated.

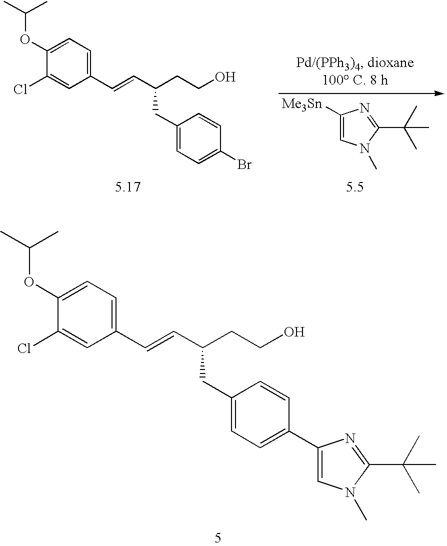

### [(3S,4E)-3-[4-(2-t-butyl-1-methyl-1H-imidazol-4-yl)benzyl]-5-(3-chloro-4-isopropoxyphenyl)-4-penten-1-ol (5)

To a pressure tube was added [(3S,4E)-3-(4-bromobenzyl)-5-(3-chloro-4-isopropoxyphenyl)-4-penten-1-ol (0.56 g, 1.3 mMol), 2-t-butyl-1-methyl-4-(trimethylstannanyl)-1H-imidazole (0.52 g, 1.7 mMol) and dioxane (10 mL). Tetrakis(triphenylphosphine)palladium(0) (80 mg, 0.07 mMol) was added, the tube purged with N₂, capped and heated to 100° C. with stirring. After 8 h at 100° C. the reaction was cooled to RT and evaporated to dryness under vacuum. Purification by flash chromatography on silica gel (50% EtOAc, hexanes) gave the title compound (192 mg, 50%) as a white solid: MS (ES) m/e 481.4 (M+H)⁺. Example 6

### Preparation of (3S)-1-(3-chloro-4-isopropoxyphenyl)-5-hydroxy-3-[4-(2-t-butyl-1-methyl-1H-imidazol-4-yl)benzyl]-1-pentanone

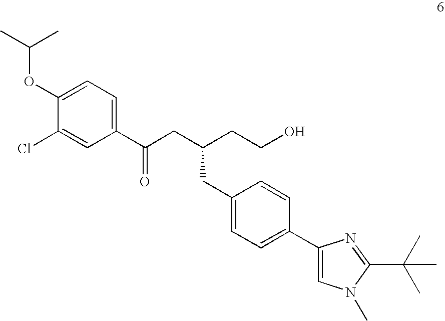 Experimental Section:

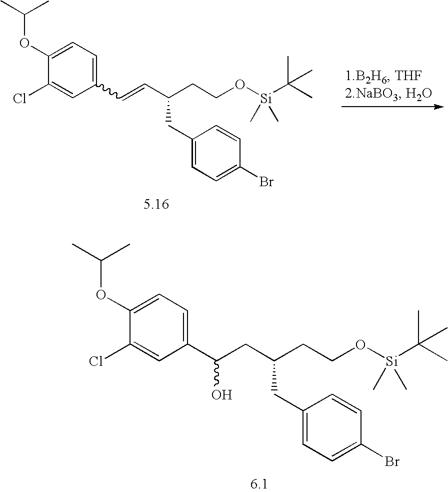

### (3S )-3-(4-bromobenzyl )-1-(3-chloro-4-isopropoxyphenyl )-5-(t-butyldimethylsilyloxy )-1-(1R,S)-pentanol (6.1)

To a stirred solution of [(3S,4E,Z)-3-(4-bromobenzyl)-5-(3-chloro-4-isopropoxyphenyl)-4-penten-1-yl]oxy]t-butyldimethylsilane (3.0 g, 5.5 mMol) in THF (6 mL) at 0° C. was added dropwise a solution of 1 N borane in THF (30 mL, 30 mMol). The reaction was allowed to slowly warm to RT and stirred for 18 h. The reaction was cooled to 0° C. then carefully quenched with H₂O (30 mL). (Vigorous gas evolution.) After stirring for 5 minutes sodium perborate tetrahydrate (6.0 g, 40 mMol) was added and stirred at 0° C. for 2 h. The reaction was diluted with H₂O, extracted with EtOAc, filtered through Celite® to remove insolubles, washed with brine, dried (Na₂SO₄), filtered and evaporated under vacuum. Purification by flash chromatography on silica gel (15% EtOAc, hexane) gave the title compound (2.10 g, 67%) as a mixture of diastereomers: MS (ES) m/e 555.2 (M+H)⁺.

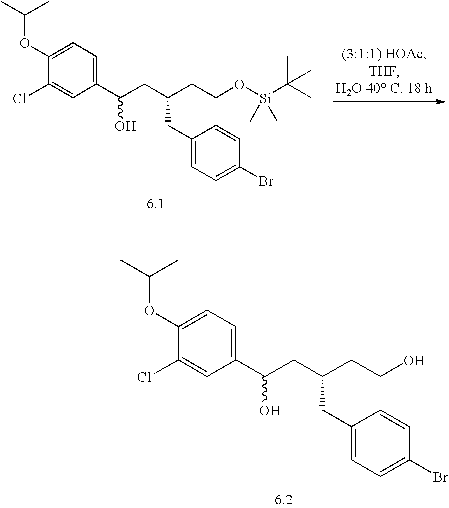

### (3S)-3-(4-bromobenzyl)-1-(3-chloro-4-isopropoxyphenyl)-5-hydroxy-(1R,S)-1-pentanol (6.2)

To (3S)-3-(4-bromobenzyl)-1-(3-chloro-4-isopropoxyphenyl)-5-(t-butyldimethylsilyloxy)-1-(1R,S)-pentanol (2.4 g, 4.3 mMol) was added a solution of (3:1:1) HOAc, THF, H₂O (20 mL). The reaction was stirred at 40° C. for 18 h, evaporated to dryness under vacuum and purified by flash chromatography on silica gel (60 to 100% EtOAc, hexanes) to give the title product (0.89 g, 93%) as a clear oil: MS (ES) m/e 423.0 (M+H—H₂O)⁺.

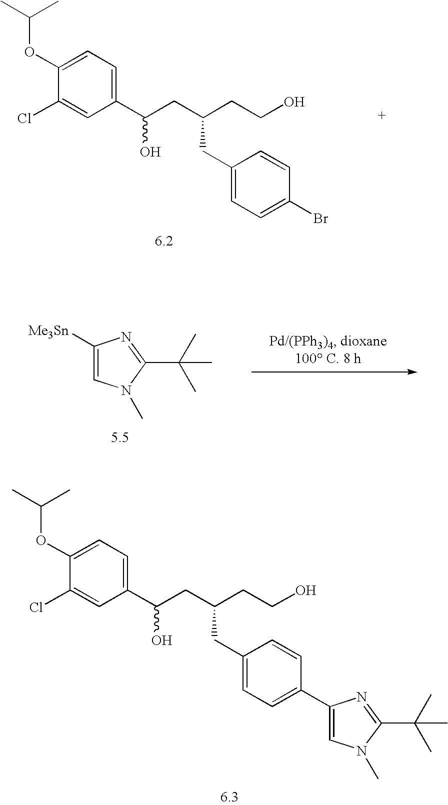

### (3S)-1-(3-chloro-4-isopropoxyphenyl)-5-hydroxy-3-[4-(2-t-butyl-1-methyl-1H-imidazol-4-yl)benzyl]-(1R,S)-1-pentanol (6.3)

To a pressure tube was added (3S)-3-(4-bromobenzyl)-1-(3-chloro-4-isopropoxyphenyl)-5-hydroxy-(1R,S)-1-pentanol (0.88 g, 2.0 mMol), 2-t-butyl-1-methyl4-(trimethylstannanyl)-1H-imidazole (0.9 g, 3.0 mMol) and dioxane (25 mL). Tetrakis(triphenylphosphine)palladium(0) (180 mg, 0.16 mMol) was added, the tube purged with N₂, capped and heated to 100° C. with stirring. After 16 h at 100° C. the reaction was cooled to RT and evaporated to dryness under vacuum. Purification by flash chromatography (EtOAc) gave the title compound (0.5 g, 50%) as a white solid foam: MS (ES) m/e 499.4 (M+H)⁺.

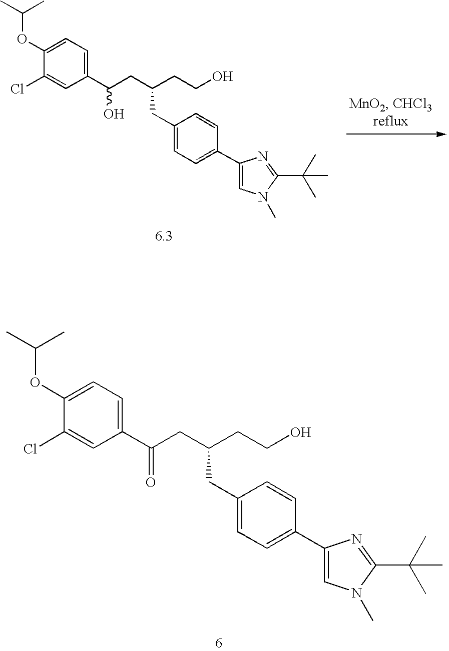

### (3S)-1-(3-chloro-4-isopropoxyphenyl)-5-hydroxy-3-[4-(2-t-butyl-1-methyl-1H-imidazol-4-yl)benzyl]-1-pentanone (6)

To a stirred solution of (3S)-1-(3-chloro-4-isopropoxyphenyl)-5-hydroxy-3-[4-(2-t-butyl-1-methyl-1H-imidazol-4-yl)benzyl]-(1R,S)-1-pentanol (0.49 g, 1.0 mMol) in CHCl₃ (25 mL) was added MnO₂ (1.0 g, 11.5 mMol). The reaction was refluxed for 18 h, cooled to RT, filtered through a pad of Celite®, rinsed with CHCl₃, and evaporated to dryness under vacuum. Purification by flash chromatography on silica gel (25 to 75% EtOAc, CH₂Cl₂) gave the title compound (242 mg, 49%) as a white solid: MS (ES) m/e 497.4 (M+H)⁺.

### Example 7

### Preparation of (1S)-1-({4-[2-(tert-butyl)-1-methyl-1H-imidazol-4-yl]phenyl}methyl)-3-hydroxypropyl 3-chloro-4-[(iso-propyl)oxy]benzoate

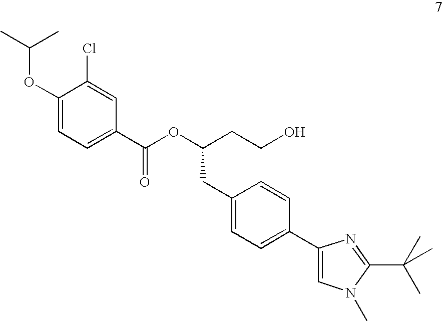 Experimental Section:

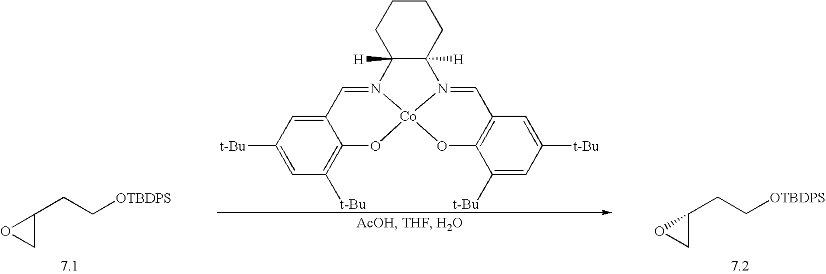

### (2R)-4-(tert-butyldiphenylsilyl)oxy-1,2-epoxybutane (7.2)

A solution of racemic 4-(tert-butyldiphenylsilyl)oxy-1,2-epoxybutane (prepared by the method of Pearson, W. H. and Fang, W.-k., J. Org. Chem., 2000, 65, 7158-7174) (6.53 g, 20.0 mmol) in tetrahydrofuran (0.2 mL) was treated with (1R,2R)-(−)-N,N′-bis(3,5-di-tert-butylsalicydene)-1,2-cyclohexanediaminocobalt(II) (0.06 g, 0.10 mmol), acetic acid (0.023 mL, 0.40 mmol), and water (0.198 mL, 11.0 mmol) at 0° C. Following removal of the cooling bath, additional tetrahydrofuran (5.0 mL) was added and the solution stirred overnight at ambient temperature. Additional water (0.198 mL, 11.0 mmol) was added and the solution stirred 3 d at ambient temperature followed by concentration in vacuo onto SiO₂. Purification via flash column chromatography (0-10% EtOAc/hexanes) gave the title compound as a light yellow oil (2.74 g; 42%). ¹H NMR (400 MHz, CHLOROFORM-d) δ ppm 7.64-7.70 (m, 4 H) 7.36-7.46 (m, 6 H) 3.76-3.90 (m, 2 H) 3.06-3.15 (m, 1 H) 2.79 (dd, J=4.9, 4.2 Hz, 1 H) 2.52 (dd, J=5.2, 2.7 Hz, 1 H) 1.78 (q, J=5.9 Hz, 2 H) 1.06 (s, 9 H). [α]_{D}=+6.82° (c=1.0, CHCl₃). MS (ES+) m/e 327 [M+H]⁺.

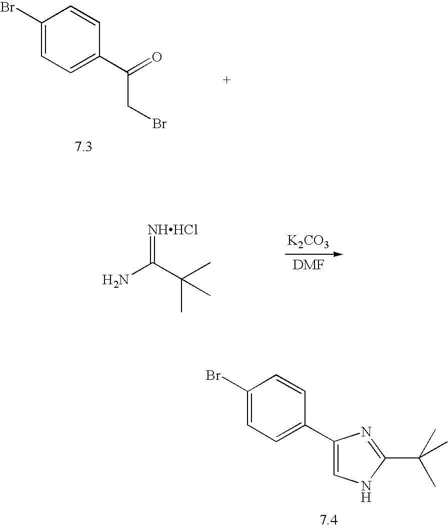

### 4-(4-bromophenyl)-2-tert-butyl-1H-imidazole (7.4)

A solution of 2,4′-dibromoacetophenone (4.17 g, 15.0 mmol) in N,N-dimethylformamide (60.0 mL) was treated with t-butylcarbamidine hydrochloride (2.05 g, 15.0 mmol) and K₂CO₃ (4.15 g, 30.0 mmol) and heated to 50° C. for 4 h. Following cooling, the reaction was quenched with water, diluted with brine, and extracted thrice with EtOAc. The organic layer was dried over MgSO₄, filtered, and concentrated in vacuo. Purification via flash column chromatography (10-40% EtOAc/hexanes) gave the title compound as a light yellow solid (3.38 g; 81%). ¹H NMR (400 MHz, CHLOROFORM-d) δ ppm 7.58 (d, J=8.1 Hz, 2 H) 7.46 (d, J=8.6 Hz, 2 H) 7.18 (s, 1 H) 1.42 (s, 9 H). MS (ES+) m/e 279/281 [M+H]⁺.

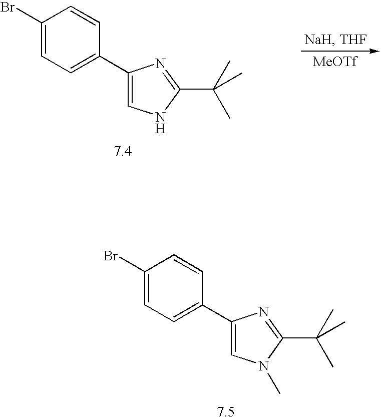

### 4-(4-bromophenyl)-2-tert-butyl-1-methyl-1H-imidazole (7.5)

A solution of 7.4 (0.718 g, 2.57 mmol) in tetrahydrofuran (15.0 mL) was treated with sodium hydride (0.123 g, 60% dispersion in mineral oil, 3.09 mmol). After stirring 15 min. at ambient temperature, methyl trifluoromethanesulfonate 0.291 mL, 2.57 mmol) was added and the solution stirred 30 min. at ambient temperature. The reaction was quenched with water, diluted with brine, and extracted twice with EtOAc. The organic layer was dried over MgSO₄, filtered, and concentrated in vacuo. Purification via flash column chromatography (5-20% EtOAc/hexanes) gave the title compound as a white solid (0.687 g; 91%). ¹H NMR (400 MHz, CHLOROFORM- [symbol] δ ppm 7.61 (ddd, J=8.8, 2.4, 2.1 Hz, 2 H) 7.44 (ddd, J=8.8, 2.4, 2.1 Hz, 2 H) 7.03 (s, 1 H) 3.77 (s, 3 H) 1.47 (s, 9 H). MS (ES+) m/e 293/295 [M+H]⁺.

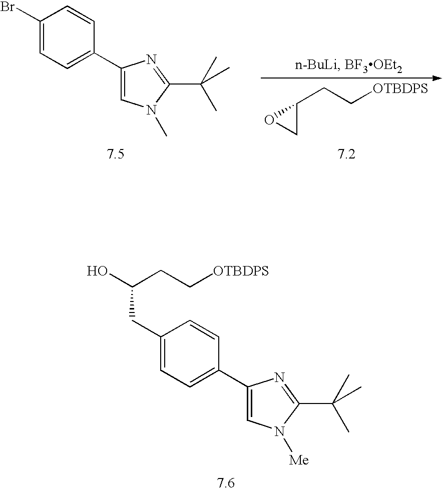

### (2S)-1-{4-[2-tert-butyl-1-methyl-1H-imidazol-4-yl]phenyl}-4-[(tert-butyldiphenylsilyl)oxy]-2-butanol (7.6)

A solution of 7.5 (0.110 g, 0.375 mmol) in tetrahydrofuran (3.0 mL) was treated with n-BuLi (0.234 mL, 1.6 M solution in hexanes, 0.375 mmol) at −78° C. After stirring 1 h at −78° C., a solution of the compound from Example 7a) (0.102 g, 0.313 mmol) in tetrahydrofuran (3.0 mL) was added, followed by boron trifluoride diethyl etherate (0.048 mL, 0.375 mmol). After stirring 2 h at −78° C., the reaction was quenched with methanol, treated with saturated aqueous NaHCO₃, and extracted thrice with EtOAc. The organic layer was dried over MgSO₄, filtered, and concentrated in vacuo. Purification via flash column chromatography (10-30% EtOAc/hexanes) gave the title compound as a light yellow oil (0.138 g; 82%). ¹H NMR (400 MHz, CHLOROFORM-d)— [symbol] ppm 7.62-7.71 (m, 6 H) 7.40 (d, J=7.6 Hz, 2 H) 7.36-7.45 (m, 4 H) 7.17 (d, J=8.3 Hz, 2 H) 7.00 (s, 1 H) 4.05-4.17 (m, 1 H) 3.86 (ddd, J=10.4, 5.2, 5.2 Hz, 1 H) 3.79-3.83 (m, 1H) 3.77 (s, 3 H) 3.12 (d, J=2.3 Hz, 1 H) 2.84 (dd, J=13.6, 6.8 Hz, 1 H) 2.74 (dd, J=13.6, 6.5 Hz, 1 H) 1.63-1.76 (m, 2 H) 1.47 (s, 9 H) 1.05 (s, 9 H). MS (ES+) m/e 541 [M+H]⁺.

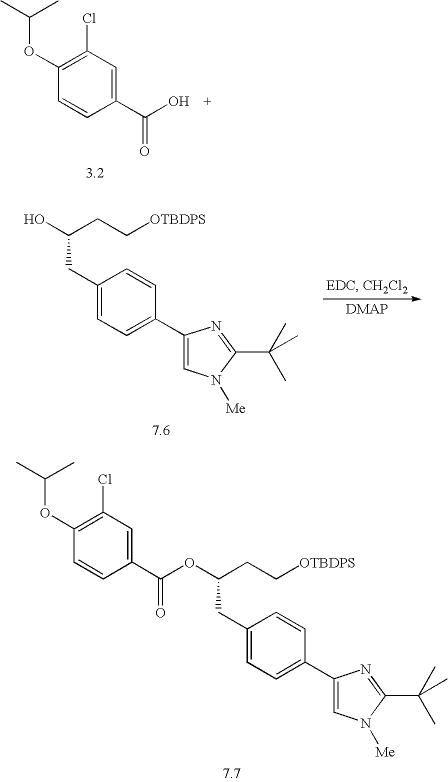

### (1S)-1-({4-[2-tert-butyl-1-methyl-1H-imidazol-4-yl]phenyl}methyl)-3-[(tert-butyldiphenylsilyl)oxy]propyl 3-chloro-4-[(iso-propyl)oxy]benzoate (7.7)

A solution of 7.6 (0.070 g, 0.129 mmol) in methylene chloride (3.0 mL) was treated with 3-chloro-4-[(1-methylethyl)oxy]benzoic acid (0.033 g, 0.155 mmol) followed by N-(3-(dimethylaminopropyl)-N′-ethylcarbodiimide (0.045 g, 0.233 mmol) and 4-dimethylaminopyridine (0.002 g, 0.013 mmol). After stirring 24 h at ambient temperature, the reaction was quenched with 1N aqueous HCl, diluted with brine, and extracted thrice with EtOAc. The organic layer was dried over MgSO₄, filtered, and concentrated in vacuo. Purification via flash column chromatography (10-40% EtOAc/hexanes) gave the title compound as a clear, colorless oil (0.076 g; 80%). MS (ES+) m/e 737 [M+H]⁺.

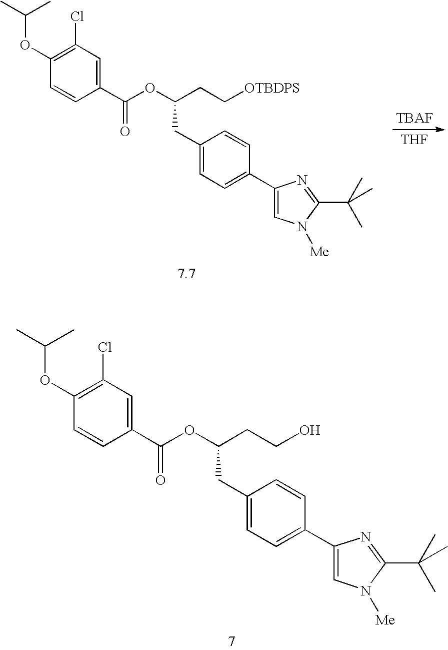

### (1S)-3-({4-[2-(tert-butyl)-1-methyl-1H-imidazol-4-yl]phenyl}methyl)-3-hydroxypropyl 3-chloro-4-[(iso-propyl)oxy]benzoate (7)

A solution of 7.7 (0.076 g, 0.103 mmol) in tetrahydrofuran (5.0 mL) was treated with tetrabutylammonium fluoride (0.206 mL, 1M solution in tetrahydrofuran, 0.206 mmol). After stirring overnight at ambient temperature, the reaction was quenched with saturated aqueous NaHCO₃ and extracted thrice with EtOAc. The organic layer was dried over MgSO₄, filtered, and concentrated in vacuo. Purification via flash column chromatography (25-50% EtOAc/hexanes) gave the title compound as a white solid (0.041 g; 80%). ¹H NMR (400 MHz, CHLOROFORM-d) δ ppm 8.03 (d, J=2.0 Hz, 1 H) 7.88 (dd, J=8.7, 2.1 Hz, 1 H) 7.69 (d, J=8.1 Hz, 2 H) 7.19 (d, J=8.3 Hz, 2 H) 7.01 (s, 1 H) 6.93 (d, J=8.8 Hz, 1 H) 4.67 (qq, J=6.1 Hz, 1 H) 4.48-4.57 (m, 1 H) 4.41 (ddd, J=11.1, 5.8, 5.6 Hz, 1 H) 3.90-4.02 (m, 1 H) 3.78 (s, 3 H) 2.85 (dd, J=13.4, 4.8 Hz, 1 H) 2.74 (dd, J=13.6, 8.0 Hz, 1 H) 1.93-2.03 (m, 1 H) 1.79-1.90 (m, 1 H) 1.48 (s, 9 H) 1.41 (d, J=6.1 Hz, 6 H). MS (ES+) m/e 499 [M+H]⁺.

### Example 8

### Preparation of (1S)-1-({4-[2-(tert-butyl)-1-methyl-1H-imidazol-4-yl]phenyl}methyl)-3-hydroxypropyl 3-cyano-4-[(iso-propyl)oxy]benzoate

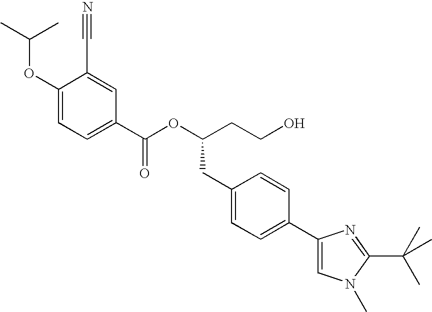

Following the procedures of Examples 7.7 and 7, except substituting 3-cyano-4-[(1-iso-propyl)oxy]benzoic acid for 3-chloro-4-[(1-iso-propyl)oxy]benzoic acid, the title compound was obtained as a white solid. ¹H NMR (400 MHz, CHLOROFORM-d) δ ppm 8.23 (d, J=2.0 Hz, 1H) 8.16 (dd, J=8.8, 2.3 Hz, 1H) 7.70 (d, J=8.1 Hz, 2 H) 7.19 (d, J=8.1 Hz, 2 H) 7.02 (s, 1 H) 6.98 (d, J=8.8 Hz, 1 H) 4.74 (qq, J=6.1 Hz, 1 H) 4.50-4.59 (m, 1 H) 4.43 (ddd, J=11.2, 6.1, 5.4 Hz, 1 H) 3.89-4.01 (m, 1 H) 3.78 (s, 3 H) 2.85 (dd, J=13.4, 4.6 Hz, 1 H) 2.75 (dd, J=13.6, 8.0 Hz, 1 H) 1.94-2.03 (m, 1 H) 1.78-1.89 (m, 1 H) 1.48 (s, 9 H) 1.44 (d, J=6.1 Hz, 6 H). MS (ES+) m/e 490 [M+H]⁺.

### Example 9

Using the procedures similar to those set forth above, the following compounds were prepared.

| | Observed |
|---|---|
| | MS |
| ChemicalName | (M + H⁺) |
| (3E)(2R)-4-[3-chloro-4-(methylethoxy)phenyl]- | 481.4 |
| N-methyl-2-[(4-phenylphenyl)methyl]but-3-enamide | |
| 2-amino-1-[3-chloro-4-(methylethoxy)phenyl]ethan- | 212.0 (M + |
| 1-ol | H⁺ − H₂O) |
| 2-amino-N-[3-chloro-4-(methylethoxy)phenyl]- | 439.0 |
| 3-[4-(phenylmethoxy)phenyl]propanamide | |
| N-[3-chloro-4-(methylethoxy)phenyl]-2- | 496.1 |
| [(methylamino)carbonylamino]-3-[4- | |
| (phenylmethoxy)phenyl]propanamide | |
| 1-[3-chloro-4-(methylethoxy)phenyl]- | 426.1 |
| 2-({[4- | |
| (phenylmethoxy)phenyl]methyl}amino)ethan-1-ol | |
| l-[3-chloro-4-(methylethoxy)phenyl]-2- | 470.5 |
| ((2-hydroxyethyl){[4- | |
| (phenylmethoxy)phenyl]methyl}amino)ethan-1-ol | |
| 2-{[(dimethylamino)sulfonyl]amino}-N- | 546.5 |
| [3-chloro-4-(methylethoxy)phenyl]- | |
| 3-[4-(phenylmethoxy)phenyl]propanamide | |
| (1S)-1-({4-[2-(tert-butyl)-1-methylimidazol- | 490 |
| 4-yl]phenyl}methyl)-3-hydroxypropyl | |
| 3-cyano-4-(methylethoxy)benzoate | |
| (1S)-1-({4-[2-(tert-butyl)-1-methylimidazol- | 499 |
| 4-yl]phenyl}methyl)-3-hydroxypropyl | |
| 3-chloro-4-(methylethoxy)benzoate | |
| (3S)-3-(acetylamino)-4-{4-[2- | 517.3 |
| (tert-butyl)imidazol-4-yl]phenyl}butyl | |
| 3-cyano-4-(methylethoxy)benzoate | |
| (3S)-3-({[3-chloro-4- | 486.2 |
| (methylethoxy)phenyl]methyl}amino)-4- | |
| {4-[2-(1-hydroxy-isopropyl)-1- | |
| methylimidazol-4-yl]phenyl}butan-1-ol | |
| (4E)(3S)-3-({4-[2-(tert-butyl)-1- | 481.4 |
| methylimidazol-4-yl]phenyl}methyl)-5-[3- | |
| chloro-4-(methylethoxy)phenyl]pent-4-en-1-ol | |
| (3S)-4-{4-[2-(tert-butyl)-1-methylimidazol- | 498.4 |
| 4-yl]phenyl}-3-({[3-chloro-4- | |
| (methylethoxy)phenyl]methyl}amino)butanoic acid | |
| (3S)-3-({4-[2-(tert-butyl)-1-methylimidazol- | 497.4 |
| 4-yl]phenyl}methyl)-1-[3-chloro-4- | |
| (methylethoxy)phenyl]-5-hydroxypentan-1-one | |
| (3S)-3-({4-[2-(tert-butyl)-1-methylimidazol- | 499.4 |
| 4-yl]phenyl}methyl)-1-[3-chloro-4- | |
| (methylethoxy)phenyl]pentane-1,5-diol | |

### Example 10

Application of a Mitotic Kinesin Inhibitor

Human tumor cells Skov-3 (ovarian) were plated in 96-well plates at densities of 4,000 cells per well, allowed to adhere for 24 hours, and treated with various concentrations of the test compounds for 24 hours. Cells were fixed in 4% formaldehyde and stained with antitubulin antibodies (subsequently recognized using fluorescently-labeled secondary antibody) and Hoechst dye (which stains DNA).

Visual inspection revealed that the compounds caused cell cycle arrest.

### Example 11

Cellular IC50s

In vitro potency of small molecule inhibitors is determined by assaying human ovarian cancer cells (SKOV3) for viability following a 72-hour exposure to a 10-point dilution series of compound. Cell viability is determined by measuring the absorbance of formnazon, a product formed by the bioreduction of MTS/PMS, a commercially available reagent. Each point on the dose-response curve is calculated as a percent of untreated control cells at 72 hours minus background absorption (complete cell kill).

Materials and Solutions:

Cells: SKOV3, Ovarian Cancer (human)
Media: RPMI medium+5% Fetal Bovine Serum+2 mM L-glutamine
Colorimetric Agent for Determining Cell Viability: Promega MTS tetrazolium compound.
Control Compound for max cell kill: Topotecan, 1 uM
   Procedure:
   Day 1—Cell Plating
   1. Wash adherent SKOV3 cells in a T175 Flask with 10 mLs of PBS and add 2 mLs of 0.25% trypsin. Incubate for 5 minutes at 37° C. Rinse cells from flask using 8 mL of media (RPMI medium+5% FBS) and transfer to fresh 50 mL sterile conical. Determine cell concentration by adding 100 uL of cell suspension to 900 uL of ViaCount reagent (Guava Technology), an isotonic diluent in a micro-centrifuge tube. Place vial in Guava cell counter and set readout to acquire. Record cell count and calculate the appropriate volume of cells to achieve 300 cells/20 uL.
   2. Add 20 ul of cell suspension (300 cells/well) to all wells of 384-well CoStar plates.
   3. Incubate for 24 hours at 37° C., 100% humidity, and 5% CO₂, allowing the cells to adhere to the plates.
      Day 2—Compound Addition
   1. In a sterile 384-well CoStar assay plate, dispense 5ul of compound at 250× highest desired concentration to wells B11-O11 (except for H11 control well) and B14-O14 (27 compounds per plate, edge wells are not used due to evaporation). 250× compound is used to ensure final uniform concentration of vehicle (DMSO) on cells is 0.4%. Dilute 14.3 ul of 10 mM Topotecan into 10 ml of 5.8% DMSO in RPMI medium giving a final concentration of 14.3 uM stock. Add 1.5 ul of this Topotecan stock to 20 ul of cell in column 13 (rows B-O) giving a final Topotecan concentration on cells of 1 uM. ODs from these wells will be used to subtract out for background absorbance of dead cells and vehicle. Add 80 ul of medium without DMSO to each compound well in column 11 and 14. Add 40 ul medium (containing 5.8% DMSO) to all remaining wells. Serially dilute compound 2-fold from column 11 to column 2 by transferring 40 ul from one column to the next taking care to mix thoroughly each time. Similarly serially dilute compound 2-fold from column 14 to column 23.
   2. For each compound plate, add 1.5 uL compound-containing medium in duplicate from the compound plate wells to the corresponding cell plates wells. Incubate plates for 72 hours at 37° C., 100% humidity, and 5% CO₂.
      Day 5—MTS Addition and OD Reading
   1. After 72 hours of incubation with drug, remove plates from incubator and add 4.5 ul MTS/PMS to each well. Incubate plates for 120 minutes at 37° C., 100% humidity, 5% CO₂. Read ODs at 490 nm after a 5 second shaking cycle in a 384-well spectrophotometer.
      For Data analysis, calculate normalized % of control (absorbance-background), and use XLfit to generate a dose-response curve. Certain chemical entities described herein showed activity when tested by this method.

### Example 12

Calculation of IC₅₀:

Measurement of a composition's IC₅₀ uses an ATPase assay. The following solutions are used: Solution 1 consists of 3 mM phosphoenolpyruvate potassium salt (Sigma P-7127), 2 mM ATP (Sigma A-3377), 1 mM IDTT (Sigma D-9779), 5 μM paclitaxel (Sigma T-7402), 10 ppm antifoam 289 (Sigma A-8436), 25 mM Pipes/KOH pH 6.8 (Sigma P6757), 2 mM MgCl2 (VWR JT400301), and 1 mM EGTA (Sigma E3889). Solution 2 consists of 1 mM NADH (Sigma N8129), 0.2 mg/ml BSA (Sigma A7906), pyruvate kinase 7 U/ml, L-lactate dehydrogenase 10 U/ml (Sigma P0294), 100 nM motor domain of a mitotic kinesin, 50 μg/ml microtubules, 1 mM DTT (Sigma D9779), 5 μM paclitaxel (Sigma T-7402), 10 ppm antifoam 289 (Sigma A-8436), 25 mM Pipes/KOH pH 6.8 (Sigma P6757), 2 mM MgCl2 (VWR JT4003-01), and 1 mM EGTA (Sigma E3889). Serial dilutions (8-12 two-fold dilutions) of the composition are made in a 96-well microtiter plate (Corning Costar 3695) using Solution 1. Following serial dilution each well has 50 μl of Solution 1. The reaction is started by adding 50 μl of solution 2 to each well. This may be done with a multichannel pipettor either manually or with automated liquid handling devices. The microtiter plate is then transferred to a microplate absorbance reader and multiple absorbance readings at 340 nm are taken for each well in a kinetic mode. The observed rate of change, which is proportional to the ATPase rate, is then plotted as a function of the compound concentration. For a standard IC₅₀ determination the data acquired is fit by the following four parameter equation using a nonlinear fitting program (e.g., Grafit 4):

$$y = \frac{Range}{1 + {( \frac{x}{{IC}_{50}} )}^{s}} + Background$$

where y is the observed rate and x the compound concentration.

Other chemical entities of this class were found to inhibit cell proliferation, although GI₅₀ values varied. GI₅₀ values for the chemical entities tested ranged from 200 nM to greater than the highest concentration tested. By this we mean that although most of the chemical entities that inhibited mitotic kinesin activity biochemically did inhibit cell proliferation, for some, at the highest concentration tested (generally about 20 μM), cell growth was inhibited less than 50%. Many of the chemical entities have GI₅₀ values less than 10 μM, and several have GI₅₀ values less than 1 μM. Anti-proliferative compounds that have been successfully applied in the clinic to treatment of cancer (cancer chemotherapeutics) have GI₅₀'s that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation at virtually any concentration may be useful.

What is claimed is:

1. At least one chemical entity chosen from compounds of Formula I 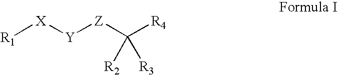
and pharmaceutically acceptable salts thereof, wherein
R₁ is chosen from optionally substituted cycloalkyl, optionally substituted aryl, optionally substituted heterocycloalkyl, and optionally substituted heteroaryl;
X is chosen from —(CR_{10 R}₁₁)ₘ, —(CR₁₀R₁₁)ₙC(R₁₃)═C(R₁₄), —O(CR₁₀R₁₁)ₚ—, and NR₈—;
Y is chosen from a direct bond linking X and Z, —C(O)—, and —C(═N—R₉)—;
Z is chosen from —(CR₁₀R₁₁)_{q}, —(CR₁₀R₁₁)ᵣC(R₁₃)═C(R₁₄), —O(CR₁₀R₁₁)ₛ—, and NR₈—;
R₈ is chosen from —CO—R₇, hydrogen, alkoxy, optionally substituted alkyl, optionally substituted cycloalkyl, optionally substituted heterocycloalkyl, sulfonyl, optionally substituted aryl, and optionally substituted heteroaryl;
R₉ is chosen from hydrogen, alkoxy, optionally substituted alkyl, optionally substituted cycloalkyl, optionally substituted heterocycloalkyl, cyano, optionally substituted aryl, and optionally substituted heteroaryl;
R₁₀ and R₁₁ are independently chosen from hydrogen, hydroxy, optionally substituted alkyl, optionally substituted aryl, optionally substituted heterocycloalkyl, or optionally substituted cycloalkyl;
R₁₃ and R₁₄ are independently chosen from hydrogen, optionally substituted alkyl, optionally substituted aryl, optionally substituted heterocycloalkyl, or optionally substituted cycloalkyl;
m is chosen from 0, 1, and 2;
n is chosen from 0 and 1;
p is chosen from 0, 1, and 2;
q is chosen from 0, 1, and 2;
r is chosen from 0 and 1;
s is chosen from 0, 1, and 2;
R₂ is chosen from hydrogen, hydroxy, optionally substituted alkoxy, optionally substituted amino, optionally substituted heterocycloalkyl, optionally substituted cycloalkyl, and optionally substituted alkyl;
R₃ and R₄ are independently chosen from hydrogen, optionally substituted alkyl, optionally substituted alkoxycarbonyl, optionally substituted amino, aminocarbonyl, optionally substituted aryl, optionally substituted heteroaryl, optionally substituted heterocycloalkyl, and optionally substituted cycloalkyl; or
R₂ and R₄, taken together with the carbon to which they are bound, form an optionally substituted 3 to 7-membered ring which optionally includes one, two, or three heteroatoms chosen from O, N, and S; or
R₂ and R₃, taken together with the carbon to which they are bound, form an optionally substituted 3 to 7-membered ring which optionally includes one, two, or three heteroatoms chosen from O, N, and S; and
R₇ is chosen from optionally substituted lower alkyl, optionally substituted aryl, hydroxy, optionally substituted amino, optionally substituted aralkoxy, or optionally substituted alkoxy,
provided that when Y is —C(O)—, and Z is NR₈, then X is not —(CR₁₀R₁₁)ₘ wherein m is 0; and provided that if Y is a direct bond linking X and Z and X is —(CR₁₀R₁₁)ₘ wherein m is 0, then Z is not —(CR₁₀R₁₁)_{q} wherein q is 0.

2. At least one chemical entity of claim 1 wherein R₁ is optionally substituted aryl.

3. At least one chemical entity of claim 2, wherein R₁ is optionally substituted phenyl.

4. At least one chemical entity of claim 3, wherein R₁ is phenyl substituted with one, two or three groups independently selected from optionally substituted heterocycloalkyl, optionally substituted alkyl, sulfonyl, halo, optionally substituted amino, sulfanyl, optionally substituted alkoxy, optionally substituted aryloxy, optionally substituted heteroaryloxy, acyl, hydroxy, nitro, cyano, optionally substituted aryl, and optionally substituted heteroaryl.

5. At least one chemical entity of claim 4, wherein R₁ is chosen from 3-halo-4-isopropoxy-phenyl, 3-cyano-4-isopropoxy-phenyl, 3-halo-4-((R)-1,1,1-trifluoropropan-2-yloxy)phenyl, 3-cyano-4-((R)-1,1,1-trifluoropropan-2-yloxy)phenyl, 3-halo-4-isopropylamino-phenyl, 3-cyano-4-isopropylamino-phenyl, 3-halo-4-((R)-1,1 1-trifluoropropan-2-ylamino)phenyl, and 3-cyano-4-((R)-1,1,1-trifluoropropan-2-ylamino)phenyl.

6. At least one chemical entity of claim 1 wherein R₂ is chosen from hydrogen, optionally substituted alkoxy, and optionally substituted amino.

7. At least one chemical entity of claim 6 wherein R₂ is hydrogen.

8. At least one chemical entity of claim 1 wherein the compound of Formula I is chosen from compounds of Formula II 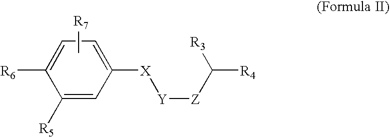
wherein
R₅ is chosen from optionally substituted heterocycloalkyl, optionally substituted lower alkyl, nitro, cyano, hydrogen, sulfonyl, and halo;
R₆ is chosen from hydrogen, halo, optionally substituted alkyl, optionally substituted cycloalkyl, optionally substituted heterocycloalkyl, optionally substituted amino, sulfanyl, optionally substituted alkoxy, cycloalkyloxy, optionally substituted heterocycloalkyloxy, optionally substituted aryloxy, and optionally substituted heteroaryloxy; and
R₇ is chosen from hydrogen, acyl, optionally substituted alkyl, optionally substituted cycloalkyl, optionally substituted alkoxy, halo, hydroxy, nitro, cyano, optionally substituted amino, sulfonyl, carboxyalkyl, aminocarbonyl, optionally substituted aryl, and optionally substituted heteroaryl.

9. At least one chemical entity of claim 1 wherein R₃ is chosen from hydrogen, aminocarbonyl, optionally substituted amino, optionally substituted lower alkyl, optionally substituted heterocycloalkyl, and optionally substituted heteroaryl.

10. At least one chemical entity of claim 9 wherein R₃ is chosen from carbamoyl, (mono-lower alkyl)carbamoyl, optionally substituted amino, and optionally substituted lower alkyl.

11. At least one chemical entity of claim 8 wherein the compound of Formula II is chosen from compounds of Formula III

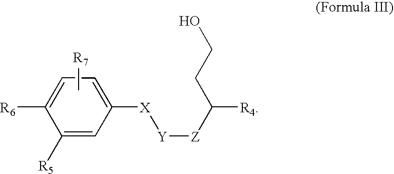

12. At least one chemical entity of claim 8 wherein the compound of Formula II is chosen from compounds of Formula IV 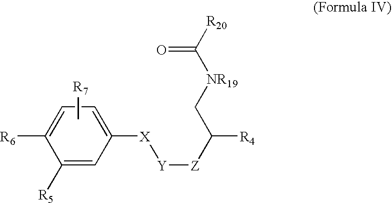
wherein
R₁₉ is chosen from hydrogen and optionally substituted lower alkyl; and
R₂₀ is chosen from optionally substituted alkyl, optionally substituted cycloalkyl, optionally substituted aryl, optionally substituted heterocycloalkyl, optionally substituted heteroaryl, optionally substituted alkoxy, and optionally substituted amino.

13. At least one chemical entity of claim 12 wherein R₁₉ is chosen from hydrogen and lower alkyl.

14. At least one chemical entity of claim 13 wherein R₁₉ is hydrogen.

15. At least one chemical entity of claim 12 wherein R₂₀ is chosen from optionally substituted alkyl, optionally substituted amino, and optionally substituted heterocycloalkyl.

16. At least one chemical entity of claim 15 wherein R₂₀ is chosen from (dimethylamino)methyl, azetidin-1-ylmethyl, pyrrolidin-1-ylmethyl, (methylamino)methyl, aminomethyl, amino, methylamino, and dimethylamino.

17. At least one chemical entity of claim 1 wherein R₄ is chosen from optionally substituted lower alkyl and optionally substituted aryl.

18. At least one chemical entity of claim 17 wherein R₄ is chosen from phenyl and benzyl, each of which is substituted with an optionally substituted heteroaryl group and each of which phenyl and benzyl is optionally further substituted with a group chosen from halo, hydroxy, and lower alkyl.

19. At least one chemical entity of claim 18 wherein R₄ is chosen from phenyl and benzyl, each of which is substituted with a heteroaryl group chosen from
7,8-dihydro-imidazo[1,2-c][1,3]oxazin-2-yl,
3a,7a-dihydro-1H-benzoimidazol-2-yl,
imidazo[2,1-b]oxazol-6-yl,
oxazol-4-yl,
5,6,7,8-tetrahydro-imidazo[1,2-a]pyridin-2-yl,
1H-[1,2,4]triazol-3-yl,
2,3-dihydro-imidazol-4-yl,
1H-imidazol-2-yl,
imidazo[1,2-a]pyridin-2-yl,
thiazol-2-yl,
thiazol-4-yl,
pyrazol-3-yl, and
1H-imidazol-4-yl, I
each of which heteroaryl groups is optionally substituted with one, two, or three groups chosen from optionally substituted lower alkyl, halo, acyl, sulfonyl, cyano, nitro, optionally substituted amino, and optionally substituted heteroaryl.

20. At least one chemical entity of claim 8 wherein the compound of Formula II is chosen from compounds of Formula V: 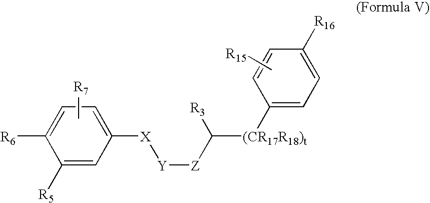
wherein
t is chosen from 0, 1, and 2;
R₁₇ and R₁₈ are independently chosen from hydrogen, hydroxy, optionally substituted alkyl, optionally substituted aryl, optionally substituted heterocycloalkyl, or optionally substituted cycloalkyl;
R₁₆ is optionally substituted heteroaryl; and
R₁₅ is chosen from hydrogen, halo, hydroxy, and lower alkyl.

21. At least one chemical entity of claim 20 wherein R₃ is chosen from hydrogen, aminocarbonyl, optionally substituted amino, optionally substituted lower alkyl, optionally substituted heterocycloalkyl, and optionally substituted heteroaryl.

22. At least one chemical entity of claim 21 wherein R₃ is chosen from carbamoyl, (mono-lower alkyl)carbamoyl, optionally substituted amino, and optionally substituted lower alkyl.

23. At least one chemical entity of claim 20, wherein R₁₆ is chosen from
7,8-dihydro-imidazo[1,2-c][1,3]oxazin-2-yl,
3a,7a-dihydro-1H-benzoimidazol-2-yl,
imidazo[2,1-b]oxazol-6-yl,
oxazol-4-yl,
5,6,7,8-tetrahydro-imidazo[1,2-a]pyridin-2-yl,
1H-[1,2,4]triazol-3-yl,
2,3-dihydro-imidazol-4-yl,
1H-imidazol-2-yl,
imidazo[1,2-a]pyridin-2-yl,
thiazol-2-yl,
thiazol-4-yl,
pyrazol-3-yl, and
1H-imidazol-4-yl,
each of which is optionally substituted with one, two, or three groups chosen from optionally substituted lower alkyl, halo, acyl, sulfonyl, cyano, nitro, optionally substituted amino, and optionally substituted heteroaryl.

24. At least one chemical entity of claim 23, wherein R₁₆ is chosen from
1H-imidazol-2-yl,
imidazo[1,2-a]pyridin-2-yl; and
1H-imidazol-4-yl,
each of which is optionally substituted with one or two groups chosen from optionally substituted lower alkyl, halo, and acyl.

25. At least one chemical entity of claim 20 wherein R₁₅ is hydrogen.

26. At least one chemical entity of claim 8 wherein R₅ is hydrogen, cyano, nitro, or halo.

27. At least one chemical entity of claim 26 wherein R₅ is chloro or cyano.

28. At least one chemical entity of claim 8 wherein R₆ is optionally substituted lower alkoxy, optionally substituted lower alkyl, or optionally substituted amino.

29. At least one chemical entity of claim 28 wherein R₆ is lower alkoxy, 2,2,2-trifluoro-1-methyl-ethoxy, lower alkylamino or 2,2,2-trifluoro-1-methyl-ethylamino.

30. At least one chemical entity of claim 29 wherein R₆ is propoxy, 2,2,2-trifluoro-1-methyl-ethoxy, propylamino, or 2,2,2-trifluoro-1-methyl-ethylamino.

31. At least one chemical entity of claim 8 wherein R₇ is hydrogen.

32. At least one chemical entity of claim 1 wherein X is —(CR₁₀R₁₁)ₘ and m is 0.

33. At least one chemical entity of claim 1 wherein X is —(CR₁₀R₁₁)ₘ and m is 1.

34. At least one chemical entity of claim 1 wherein X is —(CR₁₀R₁₁)ₘ and m is 2.

35. At least one chemical entity of claim 1 wherein X is —(CR₁₀R₁₁)ₙC(R₁₃)═C(R₁₄)— and n is 0.

36. At least one chemical entity of claim 1 wherein X is NR₈ and R₈ is hydrogen.

37. At least one chemical entity of claim 1 wherein X is —(CR₁₀R₁₁)ₘ, R₁₀ is hydrogen, R₁₁ is hydroxy, and m is 1.

38. At least one chemical entity of claim 1 wherein X is —(CHOH)CH₂—.

39. At least one chemical entity of claim 1 wherein Y is —C(O)—.

40. At least one chemical entity of claim 1 wherein Y is a direct bond linking X and Z.

41. At least one chemical entity of claim 1 wherein Z is NR₈.

42. At least one chemical entity of claim 1 wherein Z is —(CR₁₀R₁₁)_{q} and q is 0.

43. At least one chemical entity of claim 1 wherein Z is —O(CR₁₀R₁₁)ₛ and s is 0.

44. At least one chemical entity of claim 1 wherein Z is —O(CR₁₀R₁₁)ₛ and s is 1.

45. At least one chemical entity of claim 1 wherein Z is —O(CR₁₀R₁₁)ₛ and s is 2.

46. At least one chemical entity of claim 1 wherein —X—Y-Z- is chosen from —CH₂NR₈—, —CH₂C(O)NR₈—, —NR₈C(O)NR₈—, —CH₂CH₂C(O)NR₈—, —CH═CH—, —CH₂—, —CH₂CH₂—, —CH(OH)—CH₂—NR₈—, —NR₈C(O)—, —C(O)O—, —C(O)OCH₂CH₂—, —C(O)CH₂—, and —CH(OH)—CH₂—.

47. At least one chemical entity of claim 1 wherein the compound of Formula I is chosen from
(3E)(2R)-4-[3-chloro-4-(methylethoxy)phenyl]-N-methyl-2-[(4-phenylphenyl)methyl]but-3-enamide;
2-amino-1-[3-chloro-4-(methylethoxy)phenyl]ethan-1-ol;
2-amino-N-[3-chloro-4-(methylethoxy)phenyl]-3-[4-(phenylmethoxy)phenyl]propanamide;
N-[3-chloro-4-(methylethoxy)phenyl]-2-[(methylamino)carbonylamino]-3-[4-(phenylmethoxy)phenyl]propanamide;
1-[3-chloro-4-(methylethoxy)phenyl]-2-({[4-(phenylmethoxy)phenyl]methyl}amino)ethan-1-ol;
1-[3-chloro-4-(methylethoxy)phenyl]-2-((2-hydroxyethyl){[4-(phenylmethoxy)phenyl]methyl}amino)ethan-1-ol;
2-{[(dimethylamino)sulfonyl]amino}-N-[3-chloro-4-(methylethoxy)phenyl]-3-[4-(phenylmethoxy)phenyl]propanamide;
(1S)-1-({4-[2-(tert-butyl)-1-methylimidazol-4-yl]phenyl}methyl)-3-hydroxypropyl 3-cyano-4-(methylethoxy)benzoate;
(1S)-1-({4-[2-(tert-butyl)-1-methylimidazol-4-yl]phenyl}methyl)-3-hydroxypropyl 3-chloro-4-(methylethoxy)benzoate;
(3S)-3-(acetylamino)-4-{4-[2-(tert-butyl)imidazol-4-yl]phenyl}butyl 3-cyano-4-(methylethoxy)benzoate;
(3S)-3-({[3-chloro-4-(methylethoxy)phenyl]methyl}amino)-4-{4-[2-(1-hydroxy-isopropyl)-1-methylimidazol-4-yl]phenyl}butan-1-ol;
(4E)(3S)-3-({4-[2-(tert-butyl)-1-methylimidazol-4-yl]phenyl}methyl)-5-[3-chloro-4-(methylethoxy)phenyl]pent-4-en-1-ol;
(3S)-4-{4-[2-(tert-butyl)-1-methylimidazol-4-yl]phenyl}-3-({[3-chloro-4-(methylethoxy)phenyl]methyl}amino)butanoic acid;
(3S)-3-({4-[2-(tert-butyl)-1-methylimidazol-4-yl]phenyl}methyl)-1-[3-chloro-4-(methylethoxy)phenyl]-5-hydroxypentan-1-one; and
(3S)-3-({4-[2-(tert-butyl)-1-methylimidazol-4-yl]phenyl}methyl)-1-[3-chloro-4-(methylethoxy)phenyl]pentane-1,5-diol.

48. A composition comprising a pharmaceutical excipient and at least one chemical entity of claim 1.

49. A composition according to claim 48, wherein said composition further comprises a chemotherapeutic agent other than a compound of Formula I.

50. A composition according to claim 49, wherein said composition further comprises a taxane, a vinca alkaloid, or a topoisomerase I inhibitor.

51. A method of modulating CENP-E kinesin activity which comprises contacting said kinesin with an effective amount of at least one chemical entity of claim 1.

52. A method of inhibiting CENP-E which comprises contacting said kinesin with an effective amount of at least one chemical entity of claim 1.

53. A method for the treatment of a cellular proliferative disease comprising administering to a subject in need thereof at least one chemical entity of claim 1.

54. A method for the treatment of a cellular proliferative disease comprising administering to a subject in need thereof a composition according to claim 48.

55. A method according to claim 53 wherein said disease is selected from the group consisting of cancer, hyperplasias, restenosis, cardiac hypertrophy, immune disorders, and inflammation.
